(19) United States
(12) Patent Application Publication
(10) Pub. No.: US 2023/0041432 A1
(43) Pub. Date: Feb. 9, 2023

(54) COMPOSITIONS COMPRISING POLAR LIPIDS FOR MAINTAINING OR INCREASING MOBILITY AND VITALITY

(71) Applicant: FONTERRA CO-OPERATIVE GROUP LIMITED, Auckland (NZ)

(72) Inventors: Carmen Sarah Norris, Auckland (NZ); Aaron Calvin Fanning, Auckland (NZ); Linda May Schollum, Auckland (NZ); Alastair Kenneth Hugh MacGibbon, Auckland (NZ); Caroline Ann Gunn, Auckland (NZ)

(21) Appl. No.: 17/593,529

(22) PCT Filed: Dec. 20, 2019
(86) PCT No.: PCT/IB2019/061147

§ 371 Date: Sep. 20, 2021

(30) Foreign Application Priority Data
Mar. 21, 2019 (NZ) 751936

(51) Int. Cl. A61K 31/7032; A61K 31/593; A61K 33/06; A61K 31/685; A61K 31/688; A61K 31/683; A61K 31/661; A61P 21/06; A23L 33/115; A23L 2/52

(52) U.S. Cl. CPC A61K 31/7032; A23L 2/52; A23L 33/115; A61K 31/593; A61K 31/661; A61K 31/683; A61K 31/685; A61K 31/688; A61K 33/06; A61P 21/06; A23V 2002/00

(57) ABSTRACT

The present invention provides methods of maintaining or increasing mobility and/or vitality by administering a composition comprising one or more polar lipids, in addition to uses, compositions, and medicaments comprising one or more polar lipids to maintain or increase mobility and/or vitality.

### FIELD OF THE INVENTION

The present invention relates to the use of polar lipids, in particular, polar lipids derived from milk, to maintain or increase mobility and vitality. Methods for using the polar lipids and compositions comprising the polar lipids are also provided.

### BACKGROUND OF THE INVENTION

There is a progressive decline in muscle mass, strength, function and power that begins around the age of 40-45 years and accelerates in later life (Doherty. J Appl Physiol (1985). 2003 Oct;95(4): 1717-27; Janssen et al. J Appl Physiol (1985). 2000 Jul;89(1):81-8; Lindle et al. J Appl Physiol (1985). 1997 Nov;83(5): 1581-7). Clinically, age-related muscle loss is significant because it is associated with impairments in mobility and vitality and with an increased risk for falls and fractures and other common chronic metabolic diseases (Fielding et al., J Am Med Dir Assoc. 2011 May;12(4):24956; Cruz-Jentoft et al., Age Ageing. 2018 Oct 12).

Current consensus guidelines and expert opinion recommend regular exercise, particularly progressive resistance training (PRT), and an adequate intake of food and vitamins. At present, there are no pharmaceutical agents available to prevent muscle loss or functional decline.

There remains a need for methods and compositions to maintain or increase mobility and vitality.

It is an object of the present invention to go some way towards meeting this need, or to at least provide the public with a useful choice.

### SUMMARY OF THE INVENTION

In one aspect the invention relates to a method of maintaining or increasing mobility and/or vitality in a subject, the method comprising administration of a composition comprising one or more polar lipids to a subject.

In another aspect the invention relates to use of one or more polar lipids in the manufacture of a composition or medicament for maintaining or increasing mobility and/or vitality in a subject.

In a further aspect the invention relates to one or more polar lipids for use in maintaining or increasing mobility and/or vitality in a subject.

Any of the embodiments or preferences described herein may relate to any of the aspects herein alone or in combination with any one or more embodiments or preferences described herein, unless stated or indicated otherwise.

In one embodiment the method may be a method of maintaining mobility. In another embodiment the method may be a method of increasing mobility. In one embodiment the one or more polar lipids may be for use in maintaining mobility. In another embodiment the one or more polar lipids may be for use in increasing mobility.

In various embodiments maintaining or increasing mobility may comprise
a) treating or preventing sarcopenia or one or more sequelae of sarcopenia,
b) maintaining or increasing physical performance,
c) maintaining or reducing net bone loss and/or treating or preventing a condition associated with net bone loss,
d) maintaining or increasing body tone, or
e) any combination of any two or more of (a) to (d).

In one embodiment the sarcopenia may be sarcopenia of aging. In one embodiment the sarcopenia may be sarcopenia associated with the subject being sedentary. In one embodiment the method may comprise treating or preventing sarcopenia or one or more sequelae of sarcopenia in an otherwise healthy subject.

In various embodiments maintaining or increasing physical performance may comprise
a) maintaining or increasing muscle power,
b) maintaining or increasing muscle strength,
c) maintaining or increasing muscle function,
d) maintaining or increasing balance,
e) maintaining or increasing flexibility,
f) maintaining or increasing aerobic fitness,
g) maintaining or increasing aerobic endurance,
h) maintaining or increasing sports performance,
i) any combination of any two or more of (a) to (h).

In various embodiments maintaining or reducing net bone loss may comprise
a) maintaining or increasing bone mineral content,
b) maintaining or increasing bone mineral density,
c) maintaining or increasing bone mass,
d) maintaining or reducing bone turnover,
e) maintaining or reducing bone resorption, or
f) any combination of any two or more of (a) to (e).

In various embodiments maintaining or increasing body tone may comprise
a) maintaining or increasing total body lean muscle mass,
b) maintaining or increasing trunk lean muscle mass,
c) maintaining or increasing muscle cross-sectional area,
d) maintaining or increasing muscle density,
e) maintaining or reducing total body fat mass,
f) maintaining or reducing trunk fat mass,
g) or any combination of any two or more of (a) to (5).

In various embodiments the muscle cross-sectional area may be femur or tibia muscle cross-sectional area, or a combination thereof. In various embodiments the muscle density may be femur or tibia muscle density, or a combination thereof.

In various embodiments maintaining or increasing muscle power may comprise
a) increasing static squat jump height by at least about 0.82 to about 4 cm, including at least about 0.82, 0.85, 0.9 or 1 cm,
b) increasing static squat jump power-to-weight ratio by at least about 4.5 to about 10%, including at least about 4.5, 5, 6 or 7%,
c) increasing countermovement jump height by at least about 0.65 to about 4 cm, including at least about 0.65, 0.7, 0.8, 0.9 or 1 cm,
d) increasing countermovement jump power-to-weight ratio by at least about 6.75 to about 10%, including at least about 6.75, 6.8, 6.9, 7, or 7.2%,
e) increasing fast ascent peak power by at least about 3.65 to about 8%, including at least about 3.65, 3.7, 3.8, 3.9 or 4%,
f) increasing regular pace ascent power-to-weight ratio by at least about 0.1 to about 5%, including at least about 0.1, 0.5, 0.75, 1 or 1.25%,
g) increasing concentric velocity by at least about 3.35 to about 8%, including at least about 3.35, 3.5, 3.75 or 4%,
h) increasing concentric power-to-weight ratio by at least about 3.25 to about 8%, including at least about 3.25, 3.3, 3.5, 3.75 or 4%, or
i) any combination of any two or more thereof of (a) to (h),

following administration of the composition daily for a period of at least three or four months and wherein the subject is undertaking exercise on at least two days per week compared with performance of the subject before daily administration of the composition commenced.

In various embodiments maintaining or increasing muscle strength may comprise
a) increasing maximal isometric grip strength in at least one hand by at least about 2.65 to about 8%, including at least about 2.65, 2.75, 3, 3.5, or 4%,
b) increasing maximal isometric dorsiflexion strength in at least one leg by at least about 3.85 to about 15%, including at least about 3.85, 4, 5, 6, 7 or 8%,
c) increasing one-repetition maximum leg press strength by at least about 22.1 to about 30%, including at least about 22.1, 22.25, 22.5, 23 or 23.5%, or
d) any combination of any two or more of (a) to (c),

following administration of the composition daily for a period of at least three or four months and wherein the subject is undertaking exercise on at least two days per week compared with performance of the subject before daily administration of the composition commenced.

In various embodiments maintaining or increasing muscle function may comprise decreasing movement time by at least about 5 to about 30 milliseconds, including at least about 5, 10 or 12 milliseconds as determined by having the subject perform a choice stepping reaction time test and measuring the time from movement initiation to foot contact, following administration of the composition daily for a period of at least three or four months and wherein the subject is undertaking exercise on at least two days per week compared with performance of the subject before daily administration of the composition commenced.

In one embodiment maintaining or increasing balance may comprise increasing the time for which the subject is able to perform a single-leg standing balance with eyes open or closed following administration of the composition daily for a period of at least three or four months and wherein the subject is undertaking exercise on at least two days per week compared with performance of the subject before daily administration of the composition commenced.

In one embodiment maintaining or increasing flexibility may comprise increasing maximum stretch distance by at least about 1.3 to about 5 cm, including at least about 1.3, 1.5, 2 or 2.5 cm as determined by having the subject perform a sit-and-reach test following administration of the composition daily for a period of at least three or four months and wherein the subject is undertaking exercise on at least two days per week compared with performance of the subject before daily administration of the composition commenced.

In one embodiment maintaining or increasing flexibility may comprise increasing maximum stretch distance by at least about 2.6 to about 7 cm, including at least about 2.6, 2.7, 2.8, 2.9, 3, or 3.1 cm as determined by having the subject perform a sit-and-reach test following administration of the composition daily for a period of at least two months and wherein the subject is undertaking exercise on at least two days per week compared with performance of the subject before daily administration of the composition commenced.

In one embodiment maintaining or increasing aerobic fitness may comprise increasing mean step test number by at least about 4.25 to about 18%, including at least about 4.25, 4.5, 5, 7.5 or 10% as determined by having the subject perform a step test for 2 minutes following administration of the composition daily for a period of at least three or four months and wherein the subject is undertaking exercise on at least two days per week compared with performance of the subject before daily administration of the composition commenced.

In various embodiments maintaining or reducing net bone loss may comprise
a) maintaining total body bone mineral content or increasing total body bone mineral content in the subject by at least 0.01 to about 3%, including at least about 0.01%, 0.05%, 0.1% or at least about 1.5%,
b) increasing serum parathyroid hormone (PTH) concentration by less than about 0.1 to about 10%, including less than about 8.5, 7, 5 or 3%, maintaining serum PTH concentration, or decreasing serum PTH concentration by at least about 0.01 to about 3%, including at least about 0.01, 0.1, 0.5, 1 or 1.5%,
c) decreasing serum 25-hydroyxvitamin D concentration by less than about 0.01 to about 11%, including less than about 11, 10, 5 or 3%, maintaining serum 25-hydroyxvitamin D concentration or increasing serum 25-hydroyxvitamin D concentration by at least about 0.01 to about 10%, including at least about 0.01, 0.1, 2, 5 or 7.5%,
d) decreasing plasma carboxy-terminal crosslinking telopeptide of type I collagen (β-CTx) concentration by at least about 0.25 to about 20%, including at least about 0.25, 0.5, 1, 5, 10, 12% or 15%,
e) increasing plasma procollagen type 1 N-terminal propeptide (P1NP) concentration by less than about 0.1 to about 8%, including less than about 8, 7, 5, 2 or 0.1%, maintaining plasma P1NP concentration or decreasing plasma P1NP concentration by at least about 0.1 to about 10%, including at least about 0.1, 1, 3, or 5%,
f) decreasing corrected CTX-II (corrCTx-II) concentration by at least about 2.75 to about 10%, including at least about 2.75, 3, 4 or 5%, or
g) any combination of any two or more of (a) to (f)

following administration of the composition daily for a period of at least three or four months and wherein the subject is undertaking exercise on at least two days per week compared with bone mineral density or concentration measured in the subject before daily administration of the composition commenced.

In various embodiments maintaining or increasing body tone may comprise
a) decreasing total body fat mass by at least about 0.05 to about 1.5 kg, including at least about 0.05, 0.1, 0.2, 0.3, 0.4 or 0.5 kg,
b) increasing total body lean muscle mass by at least about 0.75 to about 1.5 kg, including at least about 0.75, 0.8, 0.9 or 1 kg,
c) increasing trunk lean muscle mass by at least about 0.25 to about 1 kg, including at least about 0.25, 0.3, 0.4 or 0.5 kg,
d) increasing femur muscle cross-sectional area by at least about 3.25 to about 8%, including at least about 3.25, 3.5, 3.75, 4 or 4.5%,
e) increasing femur muscle density by at least about 0.4 to about 1%, including at least about 0.4, 0.5, 0.6 or 0.7%,
f) increasing tibia muscle cross-sectional area by at least about 0.7 to about 5%, including at least about 0.7, 0.75, 0.8, 1, 1.25 or 1.5%,
g) increasing tibia muscle density by at least about 0.2 to about 1%, including at least about 0.2, 0.25, 0.3 or 0.4%, or
h) any combination of any two or more of (a) and (g)

following administration of the composition daily for a period of at least three or four months and wherein the subject is undertaking exercise on at least two days per week compared with fat mass, lean muscle mass, muscle cross-sectional area and/or muscle density measured in the subject before administration of the composition commenced.

In one embodiment the method may be a method of maintaining vitality. In another embodiment the method may be a method of increasing vitality. In one embodiment the one or more polar lipids may be for use in maintaining vitality. In another embodiment the one or more polar lipids may be for use in increasing vitality.

In various embodiments maintaining or increasing vitality may comprise
a) decreasing sedentary time by at least about 7 to about 25 minutes per day, including at least about 7, 10, 15 or 20 minutes per day,
b) increasing light intensity physical activity time by at least about 3 to about 15 minutes per day, including at least about 3, 5, 7 or 10 minutes per day,
c) increasing moderate-vigorous physical activity time by at least about 1.25 to about 10 minutes per day, including at least about 1.25, 1.5, 2.5, 3 or 5 minutes per day,
d) increasing the score of the subject on the subjective vitality scale by at least about 2.6 to about 8, including at least about 2.6, 2.75, 3, or 3.25, or
e) any combination of any two or more of (a) to (d)

following administration of the composition daily for a period of at least three or four months and wherein the subject is undertaking exercise on at least two days per week compared with time and/or score for the subject before daily administration of the composition commenced.

In one embodiment the subject may be a subject in need thereof.

In various embodiments the composition may be administered within about 1, 2, 3, 4, 5, 6, 8, 10, 12, 15, 16, 18, 20, 22 or 24 hours of the subject undertaking exercise. In various embodiments the composition may be administered before and/or after the subject undertakes exercise.

In various embodiments the subject may be aged at least about 18, 20, 21, 25, 30, 35, 40, 45, 50, 55, 60, 65, 70, 75, 80, 85, 90 or 95 years, and any ranges may be selected from between any of these values, for example, from about 18 to about 95, 18 to about 90, 20 to about 95, 20 to about 90, 25 to about 95, 25 to about 80, 30 to about 95, 30 to about 90, about 30 to about 80, about 30 to about 75, about 30 to about 70, about 30 to about 65, 35 to about 95, about 35 to about 90, 40 to about 95, about 40 to about 90, about 40 to about 80, about 40 to about 75, about 40 to about 70, about 40 to about 65, 45 to about 95, about 45 to about 90, about 45 to about 80, about 45 to about 75, about 45 to about 70, or about 45 to about 65).

In one embodiment the subject may be female.

In various embodiments the subject may be sedentary for at least about 1, 2, 3, 4, 5, 6, 7, 8, 9, 10, 12, 14, 16, 18, 20, 22 or 24 hours per day, and any ranges may be selected from between any of these values, for example, from about 1 to about 24, about 3 to about 24, about 5 to about 24, about 6 to about 24, about 7 to about 24, about 8 to about 24 or about 9 to about 24 hours.

In various embodiments the subject may have a body mass index (BMI) of greater than about 15, 16, 17, 18, 19, 20, 21, 22, 23, 24, 25, 26, 27, 28, 29, 30, 35, 40, 45 or 50 kg/m² and various ranges may be selected from between any of these values, for example, a BMI of from about 15 to about 50, about 16 to about 50, about 17 to about 50, about 20 to about 50, about 15 to about 40, about 16 to about 40 or about 17 to about 40.

In various embodiments the method may comprise administering the composition at least 1, 2, 3, or 4 times per day for a period of at least about 1, 2, 3, 4, 5 or 6 weeks, or 1, 2, 3, 4, 5, 6, 7, 8, 9, 10, 11 or 12 months, and any ranges may be selected from between any of these values, for example, from about 1 week to about 12 months, about 1 to about 12 months, about 2 to about 12 months, about 3 to about 12 months, about 4 to about 12 months or about 6 to about 12 months.

In various embodiments the subject may be undertaking exercise on at least about 1, 2, 3, 4 or 5 days per fortnight or about 1, 2, 3, 4, 5, 6 or 7 days per week, and any ranges may be selected from between any of these values, for example, from about 1 day per fortnight to about 7 days per week, about 3 days per fortnight to about 7 days per week, about 1 to about 7 days per week, about 2 to about 7 days per week or about 3 to about 7 days per week.

In various embodiments the exercise may be light intensity, moderate intensity or vigorous intensity physical activity, or any combination of any two or more thereof.

In various embodiments the duration of the exercise may be at least about 5, 10, 15, 20, 25, 30, 35, 40, 45, 50, 55, 60, 90, or 120 minutes, and any ranges may be selected from between any of these values, for example, from about 5 to about 120, about 10 to about 120, about 15 to about 120, about 20 to about 120, about 30 to about 120, about 5 to about 90, about 10 to about 90, about 15 to about 90, about 20 to about 90, about 30 to about 90, about 5 to about 60, about 10 to about 60, about 15 to about 60, about 20 to about 60, about 30 to about 60, or about 5 to about 30 minutes.

In various embodiments administration of the composition may maintain or increase mobility and/or vitality in the subject within about 1, 2, 3, or 4 weeks or 1, 2, 3, 4, 5, 6, 7, 8, 9, 10, 11, or 12 months of commencement of daily administration of the composition, and any ranges may be selected from between any of these values, for example, from about 1 week to about 12 months, about 1 to about 12, about 2 to about 12, about 3 to about 12, about 4 to about 12, about 6 to about 12, about 1 to about 9, about 2 to about 9, about 3 to about 9, about 4 to about 9, about 6 to about 9, about 1 to about 6, about 2 to about 6, about 3 to about 6, or about 4 to about 6.

In various embodiments, the one or more polar lipids may be administered in the form of a composition with a physiologically acceptable adjuvant or carrier.

In one embodiment, the composition may be a nutritional composition.

In various embodiments, the composition may be a beverage, bar, gel, cultured milk, yoghurt, custard, ice cream, cheese, crisps, chocolate, multi-layered bites, a supplement, a tablet, a capsule, confectionery or milk powder.

In various embodiments the composition may be a beverage, bar, gel, shot, cultured milk, UHT milk, yoghurt, custard, ice cream, cheese, crisps, chocolate, multi-layered bites, a supplement, a tablet, a capsule, confectionery or milk powder.

In various embodiments may be a beverage, a fruit juice, a sports drink or a milk drink.

In various embodiments, the composition may be a pharmaceutical composition or supplement and said adjuvant or carrier is a pharmaceutically acceptable adjuvant or carrier.

In various embodiments, the composition may be suitable for oral administration. In various embodiments, the composition may be suitable for parenteral administration.

In various embodiments, the composition may be formulated to provide or may be administered separately, simultaneously or sequentially with one or more of
a) at least about 5 g protein per day,
b) from about 0.1 to about 10 g lipid per day,
c) at least about 0.75 g calcium per day,
d) at least about 7.5 µg vitamin D per day, or
e) any combination of any two more of (a) to (d).

In one embodiment, the composition may be formulated to provide or may be administered separately, simultaneously or sequentially with one or more of
a) at least about 5 g protein per day,
b) from about 0.1 to about 10 g lipid per day,
c) at least about 0.75 g calcium per day, and
d) at least about 7.5 µg vitamin D per day.

In various embodiments, the composition may comprise on a dry basis
a) at least about 5% by weight protein,
b) from about 0.01 to about 20% by weight lipid,
c) at least about 1% by weight calcium,
d) at least about 10 µg vitamin D per 100 g of the composition, or
e) any combination of any two or more of (a) to (d).

In various embodiments, the composition may comprise on a dry basis
a) at least about 5% by weight protein,
b) from about 0.01 to about 20% by weight lipid,
c) at least about 1% by weight calcium, and
d) at least about 10 µg vitamin D per 100 g of the composition.

In various embodiments the composition comprising the one or more polar lipids may be administered separately, simultaneously or sequentially with one or more additional compositions. In various embodiments the additional composition may be a nutritional composition, a beverage, bar, gel, shot, cultured milk, UHT milk, yoghurt, custard, ice cream, cheese, crisps, chocolate, multi-layered bites, a supplement, a tablet, a capsule, confectionery or milk powder. In various embodiments the additional agents may include protein, lipid, calcium, vitamin D, or any combination of any two or more thereof.

In various embodiments the composition comprising one or more polar lipids may be administered separately, simultaneously or sequentially with a composition formulated to provide
a) at least about 5 g protein per day,
b) from about 0.1 to about 10 g lipid per day,
c) at least about 0.75 g calcium per day,
d) at least about 7.5 µg vitamin D per day, or
e) any combination of any two or more of (a) to (d).

In various embodiments the composition may be formulated to provide at least about 10, 25, 50, 75, 100, 120, 125, 140, 150, 160, 175, 180, 200, 220, 225, 240, 250, 260, 275, 280, 300, 320, 325, 340, 350, 360, 375, 380, 400, 425, 450, 500, 550, 600, 650, 700, 750, 850, 900, 950, 1000, 1200, 1400, 1500, 1600, 1750, 1800, 2000, 2250, 2500, 2750, 3000, 3500, 4000 or at least about 5000 mg of the one or more polar lipids per day, and various ranges may be selected from between these values, for example, from about 10 to about 5000, about 100 to about 5000, about 200 to about 5000, about 200 to about 2500, about 200 to about 2000 about 200 to about 1500, about 200 to about 1000, about 300 to about 5000 mg about 300 to about 3000, about 300 to about 2500, about 400 to about 5000, about 400 to about 4000, about 400 to about 3000, about 400 to about 2500, about 400 to about 2000 or about 400 to about 1500 mg of the one or more polar lipids per day.

In various embodiments, the one or more polar lipids may comprise polar lipids derived from non-human mammalian milk. In various embodiments, the one or more polar lipids may consist of or consist essentially of polar lipids derived from non-human mammalian milk. In various embodiments, the non-human mammalian milk may be sheep, goat, pig, mouse, water buffalo, camel, yak, horse, donkey, llama, deer or bovine milk. Preferred is bovine milk.

In various embodiments the composition may comprise one or more milk fat fractions comprising the one or more polar lipids, the milk fat fraction selected from the group comprising cream, whey cream, high fat whey, whey protein concentrate (WPC), high fat WPC, milk protein concentrate (MPC), high fat MPC, butter, ghee, by-products of anhydrous milk fat (AMF) production, buttermilk, butter serum, beta serum, sphingolipid fractions, milk fat globule membrane fractions, milk fat globule membrane lipid fractions, phospholipid fractions, and complex lipid and combinations thereof, and hydrolysates thereof.

In various embodiments the one or more polar lipids may comprise one or more polar lipids derived from non-human mammalian milk and one or more polar lipids derived from one or more plants, one or more marine oils, one or more fats and lipids produced by fermentation with microorganisms, egg, or any combination thereof. In one embodiment the one or more plant sources of polar lipids may comprise soy lecithin. In various embodiments the one or more polar lipids may comprise egg yolk or egg lecithin or any combination thereof.

In various embodiments, the one or more polar lipids may comprise one or more phospholipids, one or more gangliosides, one or more ceramides, one or more cerebrosides, one or more sphingolipids, milk fat globule membrane (MFGM) material, or any combination of any two or more thereof. In various embodiments, the method may comprise administration of milk fat globule membrane material comprising the one of more polar lipids.

In various embodiments, the one or more gangliosides may comprise one or more glycosphingolipids, GD1, GD2, GD3, GM1, GM2, GM3, one or more monosialogangliosides, one or more disialogangliosides, or one or more polysialogangliosides, or any combination of any two or more thereof. In various embodiments, the one or more gangliosides may comprise GD3 or GM3, or a combination thereof.

In various embodiments, the one or more phospholipids may comprise one or more glycerophospholipids, one or more phosphatidylcholines, one or more phosphatidylinositols, one or more phosphatidylserines, one or more phosphatidylethanolamines, one or more sphingomyelins, one or more dihydrosphingomyelins, one or more lysophospholipids, one or more phosphatidylglycerols, or any combination of any two or more thereof.

In various embodiments, at least about 60, 70, 80, 85, 90, 95, 99 or 100% of the fatty acids in the one or more phospholipids may be C14:0 or longer, each fatty acid optionally comprising one or more, two or more, three or more, or four or more double bonds in the main carbon chain of the fatty acid, and useful ranges may be selected between any of these values (for example, about 60 to about 100%). In various embodiments, the one or more phospholipids may be obtained solely from milk fat, preferably bovine milk fat. In various embodiments, the composition may comprise one or more phosphatidylcholines and one or more non-phosphatidylcholine polar lipids.

In various embodiments, the one or more lysophospholipids may comprise one or more lysophosphatidylcholines, one or more lysophosphatidylserines, one or more lysophosphatidylethanolamines, one or more lysophosphatidylinositols, or any combination of any two or more thereof. In various embodiments, the one or more sphingomyelins may comprise sphingomyelin, dihydrosphingomyelin, or a combination thereof. In various embodiments, the one or more glycerophospholipids may comprise phosphatidylglycerol.

In various embodiments, the composition may comprise phosphatidic acid.

In various embodiments, the one or more polar lipids may be obtained solely from bovine milk fat.

In various embodiments, the composition may comprise at least about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 25, 30, 35, 40, 45, 50, 55, 60, 65, or 70% by weight polar lipid on a dry basis, and useful ranges may be selected between any of these values (for example, about 0.01 to about 2, about 0.01 to about 5, about 0.01 to about 10, about 0.01 to about 15, about 0.01 to about 20, about 1 to about 5, about 1 to about 10, about 1 to about 15, about 1 to about 20, about 0.01 to about 70, about 1 to about 70%).

In various embodiments the composition may comprise at least about 0.01, 0.05, 0.075, 0.1, 0.125, 0.15, 0.175, 0.2, 0.225, 0.25, 0.275, 0.3, 0.325, 0.35 0.375, 0.4, 0.5, 0.6, 0.7, 0.8, 0.9, 1.0, 2, 3, 4, 5, 6, 7, 8, 9, 10, 15, 20, 25, 30, 35, 40, 50, 55, 60, 65 or 70 g of the one or more polar lipids per 100 g of the composition on a dry basis, and useful ranges may be selected between any of these values (for example, about 0.01 to about 70, about 0.01 to about 50, about 0.01 to about 20, about 0.01 to about 15, about 0.01 to about 10, about 0.01 to about 5, 0.01 to about 1, about 0.01 to about 0.8, about 0.01 to about 0.5, about 0.1 to about 1, about 0.1 to about 0.8, or about 0.1 to about 0.5).

In various embodiments, the composition may comprise at least about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 25, 30, 35, 40, 45, 50, 55, 60, 65, or 70% by weight phospholipids on a dry basis, and useful ranges may be selected between any of these values (for example, about 0.01 to about 2, about 0.01 to about 5, about 0.01 to about 10, about 0.01 to about 15, about 0.01 to about 20, about 1 to about 5, about 1 to about 10, about 1 to about 15, about 1 to about 20, about 0.01 to about 70, or about 1 to about 70%).

In various embodiments, the composition may comprise at least about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 25, 30, 35, 40, 45, 50, 55, 60, 65, or 70% by weight milk fat globule membrane material on a dry basis, and useful ranges may be selected between any of these values (for example, about 0.01 to about 2, about 0.01 to about 5, about 0.01 to about 10, about 0.01 to about 15, about 0.01 to about 20, about 1 to about 5, about 1 to about 10, about 1 to about 15, about 1 to about 20, about 0.01 to about 70, or about 1 to about 70%).

In various embodiments, the composition may comprise at least about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 25, 30, 35, 40, 45, 50, 55, 60, 65, or 70% by weight on a dry basis of one or more sources of polar lipids selected from the group comprising milk fat globule membrane material, high fat whey, beta serum, buttermilk, butter serum and fractions thereof and useful ranges may be selected between any of these values (for example, about 0.01 to about 2, about 0.01 to about 5, about 0.01 to about 10, about 0.01 to about 15, about 0.01 to about 20, about 1 to about 5, about 1 to about 10, about 1 to about 15, about 1 to about 20, about 0.01 to about 70, or about 1 to about 70%).

In various embodiments, the composition may comprise
a) about 0.01 to about 0.5%, preferably about 0.02 to about 0.2% by weight of one or more phosphatidylcholines,
b) about 0.005 to about 0.07%, preferably about 0.008 to about 0.05% by weight of one or more phosphatidylinositols,
c) about 0.005 to about 0.07%, preferably about 0.008 to about 0.05% by weight of one or more phosphatidylserines,
d) about 0.01 to about 0.5%, preferably about 0.02 to about 0.2% by weight of one or more phosphatidylethanolamines,
e) about 0.01 to about 0.1%, preferably about 0.02 to about 0.08% by weight sphingomyelin, 0.01 to about 0.05%, preferably 0.001 to about 0.02% by weight dihydrosphingomyelin,
f) any combination of any two or more of a) to f).

In various embodiments, the one or more polar lipids, or one or more phospholipids may comprise from about 0.01% to about 5% by weight of the total lipid in the composition.

In various embodiments, the total phospholipid in the composition may comprise
a) about 20 to about 40%, preferably about 25 to about 30% by weight of one or more phosphatidylcholines,
b) about 5 to about 20%, preferably about 7 to about 12% by weight of one or more phosphatidylinositols,
c) about 5 to about 20%, preferably about 7 to about 12% by weight of one or more phosphatidylserines,
d) about 15 to about 40%, preferably about 20 to about 30% by weight of one or more phosphatidylethanolamines,
e) about 10 to about 30%, preferably about 15 to about 20% by weight sphingomyelin,
f) about 0.5 to about 15%, preferably about 1 to about 5% by weight dihydrosphingomyelin, or
g) any combination of any two or more of a) to f).

In various embodiments, the composition may comprise from about 2 to about 10 mg gangliosides per serve. In various embodiments, the composition may be formulated to provide from about 2 to about 20 mg gangliosides per day.

In various embodiments, the composition may comprise from about 0.3 to about 6 mg gangliosides per gram of the composition.

In various embodiments, the composition may comprise from about 5 to about 35 mg per 100 g, preferably about 10 to about 20 mg per 100 g gangliosides. In various embodiments, the gangliosides may comprise 50-100% by weight GD3 and 1-60% GM3.

In various embodiments, the composition may comprise from about 0.1 to about 5 g phospholipids per serve.

In various embodiments the composition may be formulated to provide at least about 10, 25, 50, 75, 100, 120, 125, 140, 150, 160, 175, 180, 200, 220, 225, 240, 250, 260, 275, 280, 300, 320, 325, 340, 350, 360, 375, 380, 400, 425, 450, 500, 550, 600, 650, 700, 750, 850, 900, 950, 1000, 1200, 1400, 1500, 1600, 1750, 1800, 2000, 2250, 2500, 2750, 3000, 3500, 4000 or at least about 5000 mg of one or more phospholipids per day, and various ranges may be selected from between these values, for example, from about 10 to about 5000, about 100 to about 5000, about 200 to about 5000, about 200 to about 2500, about 200 to about 2000 about 200 to about 1500, about 200 to about 1000, about 300 to about 5000 mg about 300 to about 3000, about 300 to about 2500, about 400 to about 5000, about 400 to about 4000, about 400 to about 3000, about 400 to about 2500, about 400 to about 2000 or about 400 to about 1500 mg of the one or more polar lipids per day.

In various embodiments the composition may be formulated to provide at least about 0.01, 0.1, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 99 g protein per day, and useful ranges may be selected between any of these values (for example, about 0.01 to about 20, about 0.01 to about 25, about 0.01 to about 30, about 0.01 to about 40, about 0.01 to about 50, about 0.01 to about 60, about 0.01 to about 70, about 0.01 to about 80, or about 0.01 to about 99, about 1 to about 20, about 1 to about 25, about 1 to about 30, about 1 to about 40, about 1 to about 50, about 1 to about 60, about 1 to about 70, about 1 to about 80, or about 1 to about 99, about 5 to about 20, about 5 to about 25, about 5 to about 30, about 5 to about 40, about 5 to about 50, about 5 to about 60, about 5 to about 70, about 5 to about 80, or about 5 to about 99 g).

In various embodiments the composition may comprise at least about 0.01, 0.1, 0.5, 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95 or 99% by weight protein on a dry basis and useful ranges may be selected between any of these values (for example, about 0.01 to about 10, about 0.01 to about 20, about 0.01 to about 30, about 0.01 to about 40, about 0.01 to about 50, about 0.01 to about 60, about 0.01 to about 70, about 0.01 to about 80, or about 0.01 to about 99, about 1 to about 20, about 1 to about 25, about 1 to about 30, about 1 to about 40, about 1 to about 50, about 1 to about 60, about 1 to about 70, about 1 to about 80, or about 1 to about 99, about 2 to about 20, about 2 to about 25, about 2 to about 30, about 2 to about 40, about 2 to about 50, about 2 to about 60, about 2 to about 70, about 2 to about 80, or about 2 to about 99%).

In one embodiment the protein may be from an animal, plant or insect source or any combination of any two or more thereof. In one embodiment the protein may comprise casein and an additional protein source, such as animal protein, plant protein, insect protein, protein produced by fermentation with microorganisms, or any combination thereof. Suitable sources of animal protein include meat and whey protein. Suitable sources of plant protein include cereal, pulse, legume (such as pea, bean, lentil and soy protein), fruit, nut, and seed protein, or any combination of any two or more thereof.

In various embodiments of a composition containing protein as described above, the protein may be at least about 0.01, 0.1, 0.5, 5, 10, 15, 20, 25, 30, 35, 40, 45, 50, 55, 60, 65, 70, 75, 79, 81, 85, 90, 95 or 99% by weight casein and useful ranges may be selected between any of these values (for example, about 0.01 to about 10, about 0.01 to about 20, about 0.01 to about 30, about 0.01 to about 40, about 0.01 to about 50, about 0.01 to about 60, about 0.01 to about 70, about 0.01 to about 80, or about 0.01 to about 99% casein).

In various embodiments of a composition containing protein as described above, the protein may be at least about 0.01, 0.1, 0.5, 5, 10, 15, 20, 25 or 30% by weight whey protein and useful ranges may be selected between any of these values (for example, about 0.01 to about 10, about 0.01 to about 20, about 0.01 to about 30% whey protein).

In various embodiments of a composition containing protein as described above, the protein may be at least about 0.01, 0.1, 0.5, 5, 10, 15, 20, 25 or 30% by weight plant protein and useful ranges may be selected between any of these values (for example, about 0.01 to about 10, about 0.01 to about 20, about 0.01 to about 30% plant protein). Suitable sources of plant protein include cereal, pulse, legume (such as pea, bean, lentil and soy protein), fruit, nut, and seed protein, or any combination of any two or more thereof.

In various embodiments the composition may be formulated to or may be administered separately, simultaneously or sequentially with at least about 0.01, 0.1, 0.5, 1, 2, 3, 4, 5, 6, 7, 8, 9, 10, 15, 20, 25, 30, 35, 40, 45 or 50 g lipid per day and useful ranges may be selected between any of these values (for example, about 0.01 to about 5, about 0.01 to about 10, about 0.01 to about 15, about 0.01 to about 20, about 0.01 to about 30, about 0.01 to about 40, or about 0.01 to about 50, about 0.1 to about 5, about 0.1 to about 10, about 0.1 to about 15, about 0.1 to about 20, about 0.1 to about 30, about 0.1 to about 40, or about 0.1 to about 50 g).

In various embodiments the composition may comprise at least about 0.01, 0.1, 0.5, 1, 2, 3, 4, 5, 6, 7, 8, 9, 10, 15, 20, 25, 30, 35, 40, 45 or 50% by weight lipid on a dry basis and useful ranges may be selected between any of these values (for example, about 0.01 to about 5, about 0.01 to about 10, about 0.01 to about 15, about 0.01 to about 20, about 0.01 to about 30, about 0.01 to about 40, or about 0.01 to about 50%).

In one embodiment the lipid may be from an animal or plant source.

In various embodiments the composition may comprise at least about 0.01, 0.1, 0.5, 5, 10, 15, 20, 25, 30, 35, 40, 45, 50, 60 or 70% by weight carbohydrate on a dry basis and useful ranges may be selected between any of these values (for example, about 0.01 to about 10, about 0.01 to about 20, about 0.01 to about 30, about 0.01 to about 40, about 0.01 to about 50, about 0.01 to about 60, or about 0.01 to about 70%).

In various embodiments the carbohydrate may be selected from monosaccharides, disaccharides, oligosaccharides, or polysaccharides, and any combination of any two or more thereof.

In various embodiments the composition may be formulated to provide or may be administered separately, simultaneously or sequentially with at least about 0.01, 0.05, 0.1, 0.15, 0.2, 0.25, 0.3, 0.35, 0.4, 0.45, 0.5, 0.55, 0.6, 0.65, 0.7, 0.75, 0.8, 0.9, 1, 1.25, 1.5, 2, 2.5, 3, 4 or 5 g calcium per day and useful ranges may be selected between any of these values (for example, about 0.01 to about 5, about 0.01 to about 2, about 0.01 to about 1, about 0.1 to about 5, about 0.1 to about 2, about 0.1 to about 1, about 0.5 to about 5, about 0.5 to about 2, about 0.5 to about 1 g per day).

In various embodiments the composition may comprise at least about 0.01, 0.1, 0.2, 0.3, 0.4, 0.5, 0.6, 0.7, 0.8, 0.9, 1, 2, 2.5, 3, 4, 5, 6, 7, 8, 9, 10, 15, 20, 25 or 30% by weight calcium on a dry basis and useful ranges may be selected between any of these values (for example, about 0.01 to about 3%, about 0.01 to about 4%, about 0.01 to about 5%, about 0.01 to about 10%, about 0.1 to about 3%, about 0.1 to about 4%, about 0.1 to about 5%, about 0.1 to about 10%, about 0.5 to about 3%, about 0.5 to about 4%, about 0.5 to about 5%, about 0.5 to about 10%, about 1 to about 3%, about 1 to about 4%, about 1 to about 5%, or about 1 to about 10%).

In various embodiments the composition may be formulated to or may be administered separately, simultaneously or sequentially with at least about 0.1, 0.5, 1, 1.5, 2, 2.5, 3, 3.5, 4, 4.5, 5, 5.5, 6, 6.5, 7, 7.5, 8, 8.5, 9, 9.5, 10, 12, 12.5, 14, 15, 16, 18, 20, 25, 30, 40 or 50 µg vitamin D per day and useful ranges may be selected between any of these values (for example, about 0.1 to about 50, about 0.1 to about 30, about 0.1 to about 20, about 1 to about 50, about 1 to about 30, about 1 to about 20, about 5 to about 50, about 5 to about 30, about 5 to about 20 µg per day).

In various embodiments the composition may comprise at least about 0.1, 0.5, 1, 1.5, 2, 2.5, 3, 3.5, 4, 4.5, 5, 5.5, 6, 6.5, 7, 7.5, 8, 8.5, 9, 9.5, 10, 12, 12.5, 14, 15, 16, 18, 20, 25, 30, 40, 50, 60, 70, 80, 90 or 100 µg vitamin D per 100 g of the composition and useful ranges may be selected between any of these values (for example, about 0.1 to about 100, about 0.1 to about 50, about 0.1 to about 20, about 1 to about 50, about 1 to about 30, about 1 to about 20, about 5 to about 50, about 5 to about 30, about 5 to about 20 µg per 100 g of the composition).

In one embodiment the composition may have an energy content from about 1 kcal (4.184 kJ) per 100 ml to about 300 kcal (1255 kJ) per 100 ml.

In various embodiments the composition may comprise one or more strains of probiotic microorganisms.

In various embodiments the composition may comprise one or more strains of probiotic bacteria. In various embodiments the probiotic bacteria may comprise one or more Bacillus strains, Lactobacillus strains, Bifidobacterium strains, Lactococcus strains, or a combination of any two or more thereof. In various embodiments the probiotic bacteria may comprise a Lactobacillus rhamnosus strain, a Bifidobacterium lactis strain, or a combination of both Lactobacillus rhamnosus and Bifidobacterium lactis. In one embodiment the Lactobacillus rhamnosus strain is Lactobacillus HN001 (DR20™). In one embodiment the Bifidobacterium lactis strain is Bifidobacterium lactis HN019 (DR10™).

In various embodiments the probiotic microorganisms are strains of probiotic yeast. In one embodiment the probiotic yeast may comprise one or more strains of Saccharomyces.

In one embodiment the composition or formulation may comprise at least about 0.01, 0.1, 0.5, 1, 2, 3, 4, 5, 6, 7, 8, 9 or 10% by weight minerals or vitamins and useful ranges may be selected between any of these values (for example, about 0.01 to about 2, about 0.01 to about 4, about 0.01 to about 6, about 0.01 to about 8, or about 0.01 to about 10%).

In one embodiment the composition or formulation may comprise from about 0.01% to about 95% protein, from about 0.01% to about 50% lipid, from about 0.01% to about 70% carbohydrate, and from about 0.01% to about 10% minerals and vitamins.

The term “comprising” as used in this specification means “consisting at least in part of” When interpreting statements in this specification which include that term, the features, prefaced by that term in each statement, all need to be present but other features can also be present. Related terms such as “comprise” and “comprised” are to be interpreted in the same manner.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

Other aspects of the invention may become apparent from the following description which is given by way of example only and with reference to the accompanying drawings.

### DETAILED DESCRIPTION OF THE PREFERRED EMBODIMENT

The present invention recognises the beneficial effects of administration of compositions comprising polar lipids, preferably polar lipids derived from milk, on mobility and vitality, particularly in middle-aged women.

Accordingly, in a first aspect the invention provides a method of maintaining or increasing mobility and/or vitality in a subject, the method comprising administration of a composition comprising one or more polar lipids to a subject.

While various routes and methods of administration are contemplated, oral administration of polar lipids, such as in a composition suitable for oral administration, is currently preferred. It will of course be appreciated that other routes and methods of administration may be utilised or preferred in certain circumstances.

The term “oral administration” includes oral, buccal, enteral and intragastric administration.

The term “parenteral administration” includes but is not limited to topical (including administration to any dermal, epidermal or mucosal surface), subcutaneous, intravenous, intraperitoneal, and intramuscular administration.

Unless indicated otherwise, where an amount is described in terms of percent (%) by weight or w/w, it should be understood that the amount represents the % by weight or w/w on a dry basis.

A “subject” refers to a vertebrate that is a mammal, for example, a human.

As used herein the term “and/or” means “and” or “or”, or both.

As used herein “(s)” following a noun means the plural and/or singular forms of the noun.

The term “treat” and its derivatives should be interpreted in their broadest possible context. The term should not be taken to imply that a subject is treated until total recovery. Accordingly, “treat” broadly includes amelioration and/or prevention of the onset of the symptoms or severity of a particular condition.

As used herein, the term “therapeutic” and grammatical equivalents contemplate treatment, uses or administration where symptoms of reduced mobility and/or vitality are present, for example, symptoms of sarcopenia or reduced physical performance (e.g. reduced muscle mass, power, function and/or strength) or net bone loss or symptoms thereof (e.g. bone fractures, reduced bone mineral content).

It will be appreciated that the term “prophylactic” and grammatical equivalents as used herein contemplates treatment, use, administration and the like before symptoms are apparent.

It will be appreciated that treatment includes prophylactic treatment, such as for example, the prophylactic treatment of a subject, such as a subject having an expected or established increased risk of reduced mobility and/or vitality including a middle-aged or elderly subject (e.g. a subject aged from about 35 to about 95), or a sedentary subject.

It will be further appreciated that treatment includes therapeutic treatment, such as for example, treatment of one or more sequelae associated with reduced mobility or vitality, including for example, symptoms of sarcopenia, net bone loss, and/or reduced muscle strength or power.

### 1. Polar Lipids and Methods to Obtain Polar Lipids

The polar lipids are typically food grade quality or higher (e.g. generally regarded as safe (GRAS) and/or pharmaceutical grade).

In various embodiments, one or more polar lipids for use in the invention may be derived from milk fat. Milk fat is discussed comprehensively by Fox and McSweeney eds), Advanced Dairy Chemistry, Volume 2 - Lipids, 3rd Ed, Springer Science + Business Media, Inc., 2006, hereby incorporated by reference. In addition to lipids, milk fat includes vitamins, sterols, and minor components. See Chapter 1, Composition and Structure of Bovine Milk Lipids, Fox and McSweeney, for a description of naturally occurring bovine milk fat. Fractionation of milk fat is discussed by Bylund, (Ed.) Dairy processing handbook. 1995 Tetra Pak Processing Systems AB, S-221 86 Lund, Sweden, and by Illingworth, Fractionation of fats. In Physical Properties of Lipids (Marangoni A G & Narine S S, Eds), pp. 411-448. Marcel Dekker, New York (2002), and Rombaut et al, International Journal of Food Science & Technology, (2006) 41(4):435-443, all hereby incorporated by reference. Seasonal variation of milk fat is discussed by Fox and McSweeney (2006).

Examples of milk fat fractions useful as a source of polar lipids for use in the invention include cream (typically about 20 to about 40% fat by weight, preferably about 40% fat by weight), whey cream, high fat whey, whey protein concentrate (WPC), high fat WPC, milk protein concentrate (MPC), high fat MPC, butter, ghee, by-products of anhydrous milk fat (AMF) production (typically produced by phase inversion of cream or dehydration of butter), buttermilk, butter serum, beta serum, sphingolipid fractions, milk fat globule membrane fractions, milk fat globule membrane lipid fractions, phospholipid fractions, and complex lipid (lipids that yield 3 or more types of hydrolysis product per molecule) fractions, and combinations thereof, and hydrolysates thereof.

Buttermilk, butter serum, and beta serum are discussed by Bylund, G. (Ed.) Dairy processing handbook. 1995 Tetra Pak Processing Systems AB, S-221 86 Lund, Sweden; Rombaut et al, J Dairy Sci. 2005 Feb;88(2):482-8; Rombaut et al, J Dairy Sci. (2006) 89(6): 1915-25, Rombaut et al, International Journal of Food Science & Technology, (2006) 41(4):435-443, and published international application WO 2006/041316, for example, all incorporated herein by reference. Buttermilk is a term used to describe the aqueous liquid phase obtained from traditional butter production using a butter making process which may be a batch (churn) process or a continuous (Fritz) process. Buttermilk is also a term used to describe the aqueous by-product produced by the cream concentration step of the traditional method of producing AMF from cream. This traditional method involves concentration then phase inversion of cream to produce oil that is further concentrated and polished to produce AMF. Finally, buttermilk is also a term used to describe a combination of the secondary skim and beta serum by-products of a two-serum process for AMF, butter oil, or anhydrous butter oil production. In that two-serum process, the by-product from the cream concentration step is further separated to produce secondary skim and the by-product from the oil concentration step is further separated to produce beta serum. In the first two instances, the buttermilk is produced before any phase inversion has occurred. In the third instance, the buttermilk is a combination of secondary skim produced before phase inversion and beta serum produced after phase inversion. Concentration and polishing in these processes is typically achieved by centrifugation. Phase inversion is typically achieved by homogenisation. It should be understood that the source of these dairy lipid fractions may be milk or colostrum or a combination thereof.

Useful starting materials for fractionation include cream, whey, whey cream, high fat whey, whey protein concentrate (WPC), high fat WPC, milk protein concentrate (MPC), high fat MPC, butter milk, butter serum, or beta serum, from milk or colostrum or a combination thereof.

The term “beta serum” means an aqueous dairy ingredient separated from cream containing greater than 60% fat which has been through phase inversion from an oil-in -water to a water-in-oil emulsion, as described below. For example, beta serum is produced during the production of anhydrous milk fat (AMF), butter oil, or anhydrous butter oil from cream. Preferably the beta serum is dried and lactose-reduced; preferably dried beta serum is a powder.

The terms “enrich” and “enriched” mean that the fraction or composition has a higher concentration of the named component than is present in whole milk, cream, butter, buttermilk, butter serum, or beta serum, or the parent fraction from which the fraction or composition is derived. For example, a ganglioside-enriched fraction is a fraction that has a higher ganglioside concentration than whole milk, cream, butter, anhydrous milk fat, buttermilk, butter serum, or beta serum. Equally, a phospholipid-enriched fraction is a fraction that has a higher phospholipid concentration than whole milk, cream, butter, anhydrous milk fat, buttermilk, butter serum, or beta serum.

The term “fraction” means a composition that has been isolated from a source material and that is compositionally different to the source material that the fraction was isolated from. For example, a non-human mammalian milk fat fraction, such as a sheep, goat, pig, mouse, water buffalo, camel, yak, horse, donkey, llama, deer or bovine milk fat fraction, preferably a bovine milk fat fraction, differs compositionally from the naturally occurring milk fat in whole milk. In alternative embodiments the concentration in the fraction is higher than the concentration in whole milk, or in whole colostrum, or in cream from milk, or in cream from colostrum. Preferred source material useful herein includes whole milk, cream, buttermilk, butter serum, beta serum, whey, whey cream, high fat whey, whey protein concentrate (WPC), high fat WPC, milk protein concentrate (MPC), or high fat MPC from bovine milk. Preferred fractions are lipid fractions, as described herein.

Accordingly, the term “phospholipid-enriched milk fat fraction” means an isolated fraction of non-human mammalian milk fat where the phospholipid concentration of the fraction is higher than the phospholipid concentration of naturally occurring non-human mammalian milk fat. Preferably the concentration of at least one phospholipid or at least one phospholipid and at least one ganglioside in a fraction useful herein is at least about 0.5, 1, 5, 10, 15, 20, 25, 30, 35, 40, 45, 50, 55, 60, 65, 70, 75, 80, 85, 90, 95, or 100% higher than the concentration in naturally occurring non-human mammalian milk fat, and useful ranges may be selected between these values. In alternative embodiments the concentration in the fraction is higher than the concentration in whole milk, or in whole colostrum, or in cream from milk, or in cream from colostrum, or in whey, casein, whey protein concentrate, or milk protein concentrate from milk or colostrum.

Equally, the term “ganglioside-enriched milk fat fraction” means an isolated fraction of non-human mammalian milk fat where the ganglioside concentration of the fraction is higher than the phospholipid concentration of naturally occurring non-human mammalian milk fat. Preferably the concentration of at least one ganglioside or at least one ganglioside and at least one phospholipid in a fraction useful herein is at least about 0.5, 1, 5, 10, 15, 20, 25, 30, 35, 40, 45, 50, 55, 60, 65, 70, 75, 80, 85, 90, 95, or 100% higher than the concentration in naturally occurring non-human mammalian milk fat, and useful ranges may be selected between these values. In alternative embodiments the concentration in the fraction is higher than the concentration in whole milk, or in whole colostrum, or in cream from milk, or in cream from colostrum, or in whey, casein, whey protein concentrate, or milk protein concentrate from milk or colostrum.

The term “milk fat” includes mammalian milk lipids and lipid fractions, lipid hydrolysates, and lipid fraction hydrolysates. In some embodiments, milk fat may be any mammalian milk fat including but not limited to bovine, sheep, goat, pig, mouse, water buffalo, camel, yak, horse, donkey, llama, deer or human milk fat, with bovine milk fat being a preferred source. Preferred milk fats are dairy fats, particularly bovine milk fats. Preferred milk fat has one or more of palmitic acid, oleic acid, stearic acid, or myristic acid as the most abundant fatty acid(s) present, preferably palmitic, oleic, stearic and myristic acids are the most abundant fatty acids present. In particularly preferred embodiments, the milk fat, such as cream or AMF for example, has a) substantially the same percentage by weight of palmitic acid as does normal bovine milk fat (between about 23%(w/w) and about 32%(w/w), typically about 28%(w/w) - see Table 1.2, PF Fox and PLH McSweeney eds, Advanced Dairy Chemistry Volume 2 - Lipids, 3rd Ed, Springer NY, NY (2006) ISBN-10:0-387-26364-0); b) substantially the same percentage by weight of oleic acid as does normal bovine milk fat (between about 15%(w/w) and about 22%(w/w), typically about 17%(w/w) - see Fox and McSweeny ibid); c) substantially the same percentage by weight of stearic acid as does normal bovine milk fat (between about 10%(w/w) and about 15%(w/w), typically about 12%(w/w) -see Fox and McSweeny ibid); d) substantially the same percentage by weight of myristic acid as does normal bovine milk fat (between about 9%(w/w) and about 12%(w/w), typically about 1 1%(w/w) - see Fox and McSweeny ibid);e) any two of a), b), c), or d) above; f) any three of a), b), c), or d) above; g) each of a), b), c), and d) above. Preferred milk fat fractions also include cream, butter, butter milk, butter serum, beta serum, sphingolipid fractions (including sphingomyelin fractions, ceramide fractions, cerebroside fractions or ganglioside fractions, or any combination of any two or more thereof), milk fat globule membrane lipid fractions, phospholipid fractions, and complex lipid fractions, or any combination of any two or more thereof, and hydrolysates of any one or more thereof, and fractions of the hydrolysates, combinations of any two or more hydrolysates, and combinations of one or more hydrolysed and/or one or more non-hydrolysed fractions. Preferably, the milk fat comprises at least about 10, 20, 30, 40, 50, 60, 70, 80, 85, 90, 95, 99 or 100% lipid, and useful ranges may be selected between any of these values (for example, about 60 to about 100, about 70 to about 100, about 80 to about 100, about 85 to about 100, about 90 to about 100, about 95 to about 100, about 96 to about 100, about 97 to about 100, about 98 to about 100, and about 99 to about 100%, preferably about 40% or greater to about 100%).

Fractionation methods include phase inversion, interesterification, glycerolysis, solvent fractionation (such as with ethanol, water, or acetone, used alone or sequentially), supercritical fractionation (see Astaire, et al, 2003, for example), near critical fractionation (see WO 2004/066744, for example), distillation, centrifugal fractionation, suspension crystallisation, dry crystallisation, fractionation with a modifier (e.g. soaps or emulsifiers), ultra-filtration, micro-filtration, and any process for fractionation of lipid known in the art, and combinations of these methods, all as known in the art.

In various embodiments, the fractionation method is selected from solvent fractionation of cream, whey, whey cream, high fat whey, whey protein concentrate (WPC), high fat WPC, milk protein concentrate (MPC), high fat MPC, butter milk, butter serum, or beta serum, using ethanol, water, or acetone, alone or sequentially.

Polar lipids for use in the invention may be fully or partially modified, whether naturally, chemically, enzymatically, or by any other methods known in the art, including, for example, glycosylated, sialylated, esterified, phosphorylated or hydrolysed. Lipid hydrolysates may be prepared using known techniques, including but not limited to acid hydrolysis, base hydrolysis, enzymatic hydrolysis using a lipase, for example as described in Fox and McSweeney ((2006), Chapter 15 by HC Deeth and CH Fitz-Gerald), and microbial fermentation. One method of base hydrolysis includes adding 1% KOH (in ethanol) and heating for 10 minutes. Hydrolysed material may be neutralised with acetic acid or hydrochloric acid.

Milk fat globule membrane material may be isolated according to the acidification method of Kanno & Dong-Hyun, 1990 Agric. Biol. Chem., 54(11):2845-2854, and further fractionated into lipid and protein fractions by the addition of methanol, as described by Kanno et al, 1975 Agric. Biol. Chem., 39(9): 1835-1842. A phospholipid fraction may be isolated by extracting the lipid mixture with acetone according to the procedure of Pruthi et al, 1970 Indian Journal of Dairy Science, 23:248-251. Lipid residue may be further enriched in milk fat globule membrane lipids by the selective extraction of non-polar lipids with pentane.

Fractionation methods useful to produce milk fat fractions useful herein are also described in published international patent applications WO 2006/041316, WO 2007/123424, and WO 2007/123425 that are each hereby incorporated herein by reference in their entirety.

Particularly preferred milk fat fractions useful herein include those in the following tables. These fractions may be emulsions or dried, and may be powders, optionally with components including flow aids such as lactose added to improve flowability.

Table 1A

PHOSPHOLIPID AND GANGLIOSIDE FRACTIONS

| | Fraction | | | | | |
|---|---|---|---|---|---|---|
| | 1 | 2 | 3 | 4 | 5 | 6 |
| Component (%w/w) | | | | | | |
| Protein | 30.2 | 49.7 | 60.2 | <0.01 | <0.01 | 12.4 |
| MFGM | 7.5 | 11.9 | 14.4 | 0.2 | ND | ND |
| Fat | 20.6 | 35.6 | 23.1 | 94.2 | 86.8 | 90.2 |
| Polar lipids | | | | | | |
| Phospholipid | 9.7 | 14.9 | 16.0 | 31.0 | 65.7 | 66.8 |
| PC | 2.5 | 3.8 | 4.9 | 8.1 | 16.8 | 15.0 |
| PI | 0.8 | 1.1 | 1.5 | 2.8 | 5.8 | 6.0 |
| PS | 1.1 | 1.6 | 2.1 | 4.3 | 8.7 | 7.6 |
| PE | 2.8 | 4.3 | 5.4 | 11.3 | 23.6 | 21.8 |
| SM | 2.4 | 3.6 | 4.5 | 7.5 | 16.5 | 13.6 |
| Gangliosides | 0.4 | 0.7 | 1.0 | 1.2 | 2.0 | 2.0 |
| GD3 | 0.4 | 0.6 | 0.9 | 1.1 | 1.8 | 1.8 |
| Lactose | ND | 7.8 | 11.7 | 2.6 | 6.4 | 4.0 |
| Ash | ND | 5.2 | 5.9 | 3.1 | 12.1 | 9.1 |
| Moisture | 1.9 | 2.7 | 2.9 | 2.6 | 4.6 | 2.3 |

ND = not determined; <0.01 = trace amounts

Table 1B

PHOSPHOLIPID AND GANGLIOSIDE FRACTIONS

| | Fraction | | | | | |
|---|---|---|---|---|---|---|
| | 7 | 8 | 9 | 10 | 11 | 12 |
| Component (%w/w) | | | | | | |
| Protein | 0.0 | 0.0 | ND | <2% | 10.2 | 72 |
| MFGM | 0.0 | 0.0 | ND | ND | ND | 6.5 |
| Fat | 87.0 | 84.4 | 84.6 | 35.5 | 27.9 | 17 |
| Polar lipids | | | | | | |
| Phospholipid | 24.7 | 60.2 | 27.6 | 17.6 | 15.1 | 6.3 |
| PC | 8.0 | 19.2 | 3.2 | 3.1 | 2.0 | 1.7 |
| PI | 0.7 | 2.0 | 6.0 | 2.8 | 2.9 | 0.5 |
| PS | 1.0 | 2.4 | 7.3 | 3.5 | 4.0 | 0.8 |
| PE | 7.7 | 17.0 | 6.4 | 4.9 | 4.4 | 1.7 |
| SM | 6.9 | 16.7 | 3.5 | 2.8 | 1.6 | 1.6 |
| Gangliosides | 0.0 | 0.0 | 4.5 | 1.3 | 2.0 | 0.3 |
| GD3 | 0.0 | 0.0 | 4.0 | 0.6 | 1.8 | 0.3 |
| Lactose | 4.1 | 6.2 | 8.3 | 54.9 | 58.0 | ND |
| Ash | 13.3 | 7.4 | 7.0 | 5.0 | 8.3 | ND |
| Moisture | 2.2 | 2.0 | 3.7 | 3.2 | 2.8 | ND |

ND = not determined; <0.01 = trace amounts

In one embodiment the one or more polar lipids is administered as a component of a lipid composition. Preferred lipid compositions include animal, plant and marine oils and fats and lipids produced by fermentation with microorganisms. Preferred animal fats include but are not limited to dairy fats, particularly bovine milk fat, including cream.

In one embodiment the lipid composition is selected from cream, butter, ghee, a by-product of anhydrous milk fat (AMF) production (typically produced by phase inversion of cream or dehydration of butter), buttermilk, butter serum, beta serum, sphingolipid fractions, milk fat globule (or “globular”) membrane lipid-enriched fractions (including, for example, sphingolipids, ceramides, and cerebrosides), phospholipid fractions, and complex lipid fractions, CLA-enriched milk fat, CLA-enriched milk fat fractions, and any combinations of any two or more thereof, and hydrolysates thereof, and fractions of the hydrolysates, and combinations of hydrolysed and/or non-hydrolysed fractions. These fractions may be obtained from whole milk or colostrum, and any derivatives of whole milk or colostrum, including cream, cultured cream, and whey, whey cream (milk lipid obtained from whey, including acid whey or cheese whey, preferably cheese whey), high fat whey, whey protein concentrate (WPC), high fat WPC, milk protein concentrate (MPC), or high fat MPC. Cultured cream is cream from whole milk or colostrum that has been fermented with acid-producing microorganisms, preferably lactic acid bacteria.

In one embodiment the milk fat or fraction thereof is selected from cream, butter, ghee, whey, whey cream, high fat whey, whey protein concentrate (WPC), high fat WPC, milk protein concentrate (MPC), high fat MPC, a by-product of anhydrous milk fat (AMF) production (typically produced by phase inversion of cream or dehydration of butter), buttermilk, butter serum, or beta serum, and any combination of any two or more thereof.

In one embodiment the lipid composition comprises a phospholipid-enriched fraction selected from buttermilk, one or more buttermilk fractions, butter serum, one or more butter serum fractions, beta serum, one or more beta serum fractions, one or more sphingolipid fractions, one or more milk fat globule membrane lipid fractions, one or more phospholipid fractions, one or more complex lipid fractions, phospholipid-enriched whey, phospholipid-enriched whey cream, phospholipid-enriched high fat whey, phospholipid-enriched whey protein concentrate (WPC), phospholipid-enriched high fat WPC, phospholipid-enriched milk protein concentrate (MPC), phospholipid-enriched high fat MPC, and any combination of any two or more thereof.

In one embodiment the lipid composition comprises a ganglioside-enriched fraction selected from buttermilk, one or more buttermilk fractions, butter serum, one or more butter serum fractions, beta serum, one or more beta serum fractions, one or more GD3-enriched fractions of beta serum, one or more GM3-enriched fractions of beta serum, one or more GD3- and GM3-enriched fractions of beta serum, ganglioside-enriched whey, ganglioside-enriched whey cream, ganglioside-enriched high fat whey, ganglioside-enriched whey protein concentrate (WPC), ganglioside-enriched high fat WPC, ganglioside-enriched milk protein concentrate (MPC), ganglioside-enriched high fat MPC, and any combination of any two or more thereof.

In some embodiments the fraction comprises
a) about 5 to about 100% w/w lipid, or
b) about 40 to about 100% w/w lipid, or
c) about 5 to about 95% w/w lipid and about 0 to about 75% w/w protein, or
d) about 15 to about 95% w/w lipid and about 0 to about 75% w/w protein, or
e) about 5 to about 95% w/w lipid, about 0 to about 75% w/w protein, about 5 to about 85% w/w phospholipids and about 0 to about 5% w/w ganglioside, or
f) about 15 to about 95% w/w lipid, about 0 to about 65% w/w protein, about 5 to about 70% w/w phospholipids and about 0 to about 2.5% w/w ganglioside.

In some embodiments the fraction comprises at least about 0.5, 1, 2, 3, 4, 5, 10, 15, 20, 25, 30, 35, 40, 45, 50, 55, 60, 65, 70, 75, 80, 85, 90 or 95% w/w of one or more phospholipid, and useful ranges may be selected between any of these values (for example, about 0.5 to about 95%, about 0.5 to about 10%, about 5 to about 95%, about 10 to about 95%, about 15 to about 95%, about 20 to about 95%, about 25 to about 95%, about 30 to about 95%, about 35 to about 95%, about 40 to about 95%, about 45 to about 95%, about 50 to about 95%, about 10 to about 70%, about 15 to about 70%, about 20 to about 70%, about 25 to about 70%, about 30 to about 70%, about 35 to about 70%, about 40 to about 70%, about 45 to about 70%, and about 50 to about 70% w/w phospholipid).

In some embodiments the fraction comprises at least about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% w/w of one or more phospholipids selected independently from phosphatidylcholine, phosphatidylethanolamine, sphingomyelin, phosphatidylserine, and phosphatidylinositol, and useful ranges may be selected between any of these values (for example, about 0.1 to about 30%, about 0.5 to about 30%, about 1 to about 30%, about 2 to about 30%, about 3 to about 30%, about 4 to about 30%, about 5 to about 30%, about 10 to about 30%, about 15 to about 30%, about 20 to about 30%, about 0.1 to about 5%, about 0.5 to about 5%, about 1 to about 5%, about 2 to about 5%, about 3 to about 5%, about 0.1 to about 10%, about 0.5 to about 10%, about 1 to about 10%, about 2 to about 10%, about 3 to about 10%, about 4 to about 10%, about 5 to about 10%, about 6 to about 10%, about 0.1 to about 20%, about 0.5 to about 20%, about 1 to about 20%, about 2 to about 20%, about 3 to about 20%, about 4 to about 20%, about 5 to about 20%, about 10 to about 20%, about 15 to about 20% w/w of one or more phospholipids selected independently from phosphatidylcholine, phosphatidylethanolamine, sphingomyelin, phosphatidylserine, and phosphatidylinositol).

In some embodiments the fraction comprises at least about 0, 1, 2, 3, 4, 5, 6, 7, 8, 9, 10, 11, 12, 13, 14, or 15% w/w of one or more gangliosides, and useful ranges may be selected between any of these values (for example, about 0 to about 10%, about 0 to about 15%, about 1 to about 10%, or about 1 to about 15%).

In some embodiments the fraction comprises
a) about 25 to about 35% w/w protein, about 15 to about 25% w/w lipid, and about 5 to about 12% w/w phospholipid, or
b) about 25 to about 35% w/w protein, about 15 to about 25% w/w lipid, about 5 to about 12% w/w phospholipid, about 5 to about 10% w/w MFGM protein, and about 0.2 to about 0.9% w/w ganglioside, or
c) about 25 to about 35% w/w protein, about 15 to about 25% w/w lipid, about 5 to about 12% w/w phospholipid, about 1 to about 5% w/w phosphatidylcholine, about 1.5 to about 6% w/w phosphatidylethanolamine, about 1 to about 5% w/w sphingomyelin, about 0.5 to about 2% w/w phosphatidylserine, about 0.1 to 2% w/w phosphatidylinositol, about 5 to about 10% w/w MFGM protein, and about 0.2 to about 0.9% w/w ganglioside, or
d) about 40 to about 60% w/w protein, about 25 to about 45% w/w lipid, and about 10 to about 25% w/w phospholipid, or
e) about 40 to about 60% w/w protein, about 25 to about 45% w/w lipid, about 10 to about 25% w/w phospholipid, about 5 to about 20% w/w MFGM protein, and about 0.5 to about 2.0% w/w ganglioside, or
f) about 46 to about 52% w/w protein, about 28 to about 40% w/w lipid, about 11 to about 16% w/w phospholipid, about 2 to about 6% w/w phosphatidylcholine, about 3 to about 8% w/w phosphatidylethanolamine, about 2.5 to about 7% w/w sphingomyelin, about 0.5 to about 3% w/w phosphatidylserine, about 0.5 to 2% w/w phosphatidylinositol, about 5 to about 15% w/w MFGM protein, and about 0.5 to about 0.9% w/w ganglioside, or
g) about 50 to about 70% w/w protein, about 12 to about 32% w/w lipid, and about 5 to about 25% w/w phospholipid, or
h) about 50 to about 70% w/w protein, about 12 to about 32% w/w lipid, about 5 to about 25% w/w phospholipid, about 2 to about 8 % w/w phosphatidylcholine, about 2 to about 10% w/w phosphatidylethanolamine, about 2 to about 8% w/w sphingomyelin, and about 1 to about 3% w/w phosphatidylserine, about 10 to about 20% w/w MFGM protein, and about 0.5 to about 2.5% w/w ganglioside, or
i) about 56 to about 65% w/w protein, about 18 to about 28 % w/w lipid, about 8 to about 20% w/w phospholipid, about 2 to about 8 % w/w phosphatidylcholine, about 2 to about 10% w/w phosphatidylethanolamine, about 2 to about 8% w/w sphingomyelin, and about 1 to about 3% w/w phosphatidylserine, and about 0.5 to 3% w/w phosphatidylinositol, about 10 to about 20% w/w MFGM protein, and about 0.5 to about 2.5% w/w ganglioside, or
j) about 0 to about 10% w/w protein, about 85 to about 97% w/w lipid, and about 25 to about 35% w/w phospholipid, or
k) about 0 to about 10% w/w protein, about 85 to about 97% w/w lipid, about 25 to about 35% w/w phospholipid, about 5 to about 10 % w/w phosphatidylcholine, about 7 to about 13% w/w phosphatidylethanolamine, about 4 to about 9% w/w sphingomyelin, about 2 to about 5% w/w phosphatidylserine, about 1 to about 3% w/w phosphatidylinositol, about 0 to
about 5 % w/w MFGM protein, and about 1 to about 3% w/w gangliosides, or 1) about 10 to about 15% w/w protein, about 80 to about 95% w/w lipid, and about 60 to about 80% w/w phospholipid, or
m) about 10 to about 15% w/w protein, about 80 to about 95% w/w lipid, about 60 to about 80% w/w phospholipid, about 10 to about 20 % w/w phosphatidylcholine, about 18 to about 28% w/w phosphatidylethanolamine, about 10 to about 20% w/w sphingomyelin, about 4 to about 12% w/w phosphatidylserine, about 2 to about 10% w/w phosphatidylinositol, about 0 to about 5% w/w MFGM protein, and about 1 to about 5% w/w gangliosides, or
n) about 75 to about 99% w/w lipid and about 15 to 35% w/w phospholipid, or
o) about 80 to about 90% w/w lipid, about 5 to about 15% w/w phosphatidylcholine, about 5 to about 15% w/w phosphatidylethanolamine, about 4 to about 10% w/w sphingomyelin, and about 0.1 to about 2% w/w phosphatidylserine, or
p) about 80 to about 90% w/w lipid, about 20 to 30% w/w phospholipid, about 5 to about 15% w/w phosphatidylcholine, about 5 to about 15% w/w phosphatidylethanolamine, about 5 to about 10% w/w sphingomyelin, about 0.5 to about 1.5% w/w phosphatidylserine, and about 0.1 to about 1.2% w/w phosphatidylinositol, or
q) about 75 to about 95% w/w lipid and about 50 to about 90% w/w phospholipid, or
r) about 80 to about 90% w/w lipid, about 10 to about 30% w/w phosphatidylcholine, about 12 to about 22% w/w phosphatidylethanolamine, about 12 to about 22% w/w sphingomyelin, and about 1 to about 3% w/w phosphatidylserine, or
s) about 75 to about 95% w/w lipid, about 50 to about 90% w/w phospholipid, about 10 to about 45% w/w phosphatidylcholine, about 12 to about 25% w/w phosphatidylethanolamine, about 12 to about 25% w/w sphingomyelin, about 1 to about 6% w/w phosphatidylserine, and about 0.5 to 4% w/w phosphatidylinositol, or
t) about 80 to about 90% w/w lipid, about 65 to about 75% w/w phospholipid, about 10 to about 30% w/w phosphatidylcholine, about 12 to about 22% w/w phosphatidylethanolamine, about 12 to about 22% w/w sphingomyelin, about 1 to about 3% w/w phosphatidylserine, and about 0.5 to 3% w/w phosphatidylinositol, or
u) about 25 to about 45% w/w lipid and about 0.2 to about 1% w/w ganglioside GD3, or
v) about 25 to about 45% w/w lipid, about 10 to about 30% w/w phospholipids, and about 0.2 to about 1% w/w ganglioside, or
w) about 25 to about 45% w/w lipid, about 10 to about 30% w/w phospholipids, about 2 to about 5% w/w phosphatidylcholine, about 3 to about 7% w/w phosphatidylethanolamine, about 2 to about 5% w/w sphingomyelin, about 2 to about 12% w/w phosphatidylserine, about 1 to about 5% w/w phosphatidylinositol, and about 0.2 to about 1% w/w ganglioside, or
x) about 20 to about 40% w/w lipid and about 0.8 to about 2% w/w ganglioside GD3, or
y) about 20 to about 40% w/w lipid, about 5 to about 30% w/w phospholipids, and about 0.8 to about 3.5% w/w ganglioside, or
z) about 20 to about 40% w/w lipid, about 5 to about 30% w/w phospholipids, about 1 to about 5% w/w phosphatidylcholine, about 2 to about 8% w/w phosphatidylethanolamine, about 0.5 to about 5% w/w sphingomyelin, about 1 to about 10% w/w phosphatidylserine, about 1 to about 6% w/w phosphatidylinositol, and about 0.8 to about 3.5% w/w ganglioside.

In various embodiments, the fraction may comprise at least about 30% total lipids, at least about 0.5% ganglioside GD3, and at least about 0.4% ganglioside GM3.

In another embodiment, the fraction may comprise at least about 30% total lipids, at least about 1.2% ganglioside GD3, and at least about 0.2% ganglioside GM3.

In one embodiment a composition useful herein comprises, consists essentially of, or consists of at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of one or more lipid compositions described above and useful ranges may be selected between any of these foregoing values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%).

In one embodiment a composition useful herein comprises, consists essentially of, or consists of at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of one or more lipid compositions described above and useful ranges may be selected between any of these foregoing values (for example, from about 0.01 to about 1 grams, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams).

### 2. Compositions

A composition useful herein may be formulated as, or may be administered separately, simultaneously or sequentially with, a food, drink, food additive, drink additive, dietary supplement, nutritional composition, medical food, enteral or parenteral feeding product, meal replacement, cosmeceutical, nutraceutical, or pharmaceutical. Appropriate formulations may be prepared by an art skilled worker with regard to that skill and the teaching of this specification.

In various embodiments, compositions useful herein may include any edible consumer product which is able to carry lipid. Examples of suitable edible consumer products include powders, liquids, confectionery products including chocolate and chewable confectionery such as gums, gels, ice creams or frozen confections, reconstituted fruit products, snack bars, food bars, muesli bars, spreads, sauces, dips, dairy products including yoghurts and cheeses, drinks including dairy and non-dairy based drinks (such as milk drinks and yoghurt drinks), milk powders, sports supplements including dairy and non-dairy based sports supplements, food additives such as protein sprinkles, dietary supplement products including daily supplement tablets. Suitable nutraceutical compositions useful herein may be provided in similar forms.

Examples of formulas, in powder or liquid form, include the following. It should be understood that the following formulations are indicative only and variations may be made according to known principles for formulating such products. For example, non-dairy sources of protein may be supplemented for the dairy proteins listed. Equally, hypoallergenic embodiments of these products may be provided where the protein source is fully or partially hydrolysed. Such hydrolysates are known in the art.

One example of a powdered nutritional composition useful herein may comprise
a) from about 15 to about 50 g protein per 100 g,
b) from about 0.01 to about 20 g fat per 100 g,
c) from about 20 to about 75 g carbohydrate per 100 g,
d) from about 200 mg to about 2 g polar lipids per 100 g,
e) from about 0 to about 75 µg vitamin D per 100 g,
f) from about 0.5 to about 10 g calcium per 100 g, and
g) vitamin and mineral premixes.

In one embodiment the composition comprising one or more polar lipids may be administered separately, simultaneously or sequentially with a powdered nutritional composition. For example, a powdered nutritional composition comprising
a) from about 15 to about 50 g protein per 100 g,
b) from about 0.01 to about 20 g fat per 100 g,
c) from about 20 to about 75 g carbohydrate per 100 g,
d) from about 0 to about 75 µg vitamin D per 100 g,
e) from about 0.5 to about 10 g calcium per 100 g, and
f) vitamin and mineral premixes.

In one embodiment a composition useful herein comprises, consists essentially of, or consists of about 0.1, 0.5, 1, 5, 10, 15, 20, 25, 30, 35, 40, 45, 50, 55, 60, 65, 70, 75, 80, 85, 90, 95, 97, 99, or 99.9 % by weight of fresh whole milk or a milk derivative and useful ranges may be selected between any of these foregoing values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, and from about 45 to about 50%). The milk derivative is preferably selected from recombined, powdered or fresh skim milk, recombined or reconstituted whole or skim milk powder, skim milk concentrate, skim milk retentate, concentrated milk, ultrafiltered milk retentate, milk protein concentrate (MPC), high fat MPC, milk protein isolate (MPI), calcium depleted milk protein concentrate (MPC), low fat milk, low fat milk protein concentrate (MPC), casein, caseinate, milk fat, cream, butter, ghee, anhydrous milk fat (AMF), buttermilk, butter serum, beta serum, hard milk fat fractions, soft milk fat fractions, sphingolipid fractions, milk fat globule membrane fractions, milk fat globule membrane lipid fractions, phospholipid fractions, complex lipid fractions, colostrum, a colostrum fraction, colostrum protein concentrate (CPC), colostrum whey, an immunoglobulin fraction from colostrum, whey (including sweet whey, lactic acid whey, mineral acid whey, or reconstituted whey powder), whey protein isolate (WPI), whey protein concentrate (WPC), whey cream, high fat whey, high fat WPC, a composition derived from any milk or colostrum processing stream, a composition derived from the retentate or permeate obtained by ultrafiltration or microfiltration of any milk or colostrum processing stream, a composition derived from the breakthrough or adsorbed fraction obtained by chromatographic (including but not limited to ion and gel permeation chromatography) separation of any milk or colostrum processing stream, extracts of any of these milk derivatives including extracts prepared by multistage fractionation, differential crystallisation, solvent fractionation, supercritical fractionation, near critical fractionation, distillation, centrifugal fractionation, or fractionation with a modifier (e.g. soaps or emulsifiers), hydrolysates of any of these derivatives, protein hydrolysates, fractions of the hydrolysates, and any combination of any two or more of these derivatives, including combinations of hydrolysed and/or non-hydrolysed fractions. It should be understood that the source of these derivatives may be milk or colostrum or a combination thereof.

In various embodiments the composition comprises one or more glycerides (including one or more monoglycerides, one or more diglycerides, or one or more triglycerides, or any combination of any two or more thereof), one or more ceramides, one or more ether glycerophospholipids, one or more cerebrosides (including one or more glucosylceramides or one or more lactosylceramides, or combinations thereof), or one or more sulfatides, or any combination of any two or more thereof.

Formulas useful herein may also comprise 0.1 to 4% w/w, preferably 2 to 4% w/w of one or more of a vitamin premix, a mineral premix, lecithin, one or more antioxidants, one or more stabilisers, or one or more nucleotides, or any combination of any two or more thereof. In some embodiments, formulas may be formulated to provide about 1000 to about 2000 kJ/100 g, or about 2700 and about 3000 kJ/L.

Examples of edible consumer products of the invention, such as yoghurts or dairy based drinks (such as milk drinks and yogurt drinks) or a concentrated dairy “shot” will typically comprise and may consist of a protein source (such as a dairy protein source), a lipid source, a carbohydrate source, in addition to the one or more polar lipids. Flavourants, colourants, and other additives, carriers or excipients as are well known to those skilled in the art may also be included.

Example formulations for edible consumer products suitable for use in the invention are provided in the following table.

| | | Product | |
|---|---|---|---|
| | Yoghurt | UHT | Shot |
| Ingredient | % by weight | % by weight | % by weight |
| Water | 83.86 | 86.37 | 75.11 |
| Skim milk powder | 10.1 | 8.45 | 16.9 |
| Buttermilk powder | 2.7 | | 0 |
| MFGM | 0.74 | 1.36 | 2.72 |
| Whole milk powder | 0.6 | 0.62 | 1.24 |
| Mineral premix | 0.17 | | |
| Collagen | 0.055 | 0.0574 | 0.1148 |
| Calcium salt | 0.25 | 0.44 | 0.88 |
| Yoghurt culture | 0.001 | | |
| Milk protein concentrate | | 2.04 | 2.04 |
| Yoghurt stabilisers | 1.5 | 0.1 | 0.1 |
| Stabiliser | | 0.24 | 0.24 |
| Magnesium salt | | 0.2805 | 0.561 |
| Zinc salt | | 0.0008 | 0.0016 |
| Vitamin premix | 0.017 | 0.046 | 0.092 |
| Ingredient | % by weight | % by weight | % by weight |
| Serving size | 200q | 250ml | 125ml |

A further example of an edible consumer product amenable to use in the present invention is the Unistraw™ delivery system (Unistraw International Limited, Australia) as described in PCT international application PCT/AU2007/000265 (published as WO 2007/098564) and PCT international application PCT/AU2007/001698 (published as WO 2008/055296), each incorporated herein in its entirety. It will be appreciated by those skilled in the art that one or more polar lipids may be coated onto a substrate (for example, a water soluble bead) for use in such delivery systems.

In alternative embodiments, the compositions useful herein may be formulated to allow for administration to a subject by any chosen route, including but not limited to oral or parenteral (including topical, subcutaneous, intramuscular and intravenous) administration.

For example, a nutraceutical composition for use according to the invention can be a dietary supplement (e.g., a capsule, a mini-bag, or a tablet) or a food product (e.g., milk, juice, a soft drink, a herbal tea-bag, or confectionery). The composition can also include other nutrients, such as a protein, a carbohydrate, vitamins, minerals, amino acids or peptides. The composition can be in a form suitable for oral use, such as a tablet, a hard or soft capsule, an aqueous or oil suspension, or a syrup; or in a form suitable for parenteral use, such as an aqueous propylene glycol solution, or a buffered aqueous solution. The amount of the active ingredient in the nutraceutical composition depends to a large extent on a subject’s specific need. The amount also varies, as recognized by those skilled in the art, dependent on administration route, and possible co-usage of other probiotic factors or probiotic agents.

It will be appreciated that in certain embodiments, the compositions of the invention may be formulated so as to have a desired calorific content, for example so as to deliver a desired amount of energy or a desired percentage of daily recommended energy intake. For example, an edible consumer product may be formulated to provide from about 200 to about 2000 kJ per serve, or from about 500 kJ to about 2000 kJ per serve, or from about 750 to about 2000 kJ per serve.

Additionally, it is contemplated that a composition in accordance with the invention may be formulated with additional active ingredients which may be of benefit to a subject in particular instances. For example, agents, such as therapeutic agents that target the same or different facets of the condition or disease process or interest may be used.

In one embodiment the additional active ingredients may include lipids, carbohydrates, other proteins, minerals or vitamins, or flavouring agents.

In one embodiment the composition may additionally comprise a source of amino acids, such as free amino acids, or peptides as described above.

In one embodiment the composition may be supplemented with minerals, including, but not limited to chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

The compositions may also optionally comprise vitamins. The vitamins may be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

It should be understood that the additional agents may also be employed in a method according to the invention where they are administered separately, simultaneously or sequentially with a composition useful herein. The proportions of each component in the composition are tailored to optimise the efficacy of the composition for maintaining or increasing mobility and/or vitality in the subject.

Thus, a pharmaceutical composition useful according to the invention may be formulated with an appropriate pharmaceutically acceptable carrier (including excipients, diluents, auxiliaries, and combinations thereof) selected with regard to the intended route of administration and standard pharmaceutical practice. For example, a composition useful according to the invention can be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

The term “pharmaceutically acceptable carrier” is intended to refer to a carrier including but not limited to an excipient, diluent or auxiliary, pharmaceutically acceptable carrier includes a solvent, a dispersion medium, a coating, an antibacterial and antifungal agent, and an isotonic and absorption delaying agent or combination thereof, that can be administered to a subject as a component of a composition described herein that does not reduce the activity of the composition and is not toxic when administered in doses sufficient to deliver an effective amount of a compound or composition useful herein. The formulations can be administered orally, nasally or parenterally (including topically, intramuscularly, intraperitoneally, subcutaneously and intravenously).

In certain embodiments, a composition of the invention (such as, for example, a nutraceutical or pharmaceutical composition of the invention, may be provided as a capsule. Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite. Active ingredients can also be administered in a form of a hard shell tablet or a capsule containing a binder, e.g., lactose or mannitol, a conventional filler, and a tabletting agent. Pharmaceutical compositions can also be administered via the parenteral route. Examples of parenteral dosage forms include aqueous solutions, isotonic saline or 5% glucose of the active agent, or other well-known pharmaceutically acceptable excipients. Cyclodextrins, or other solubilising agents well-known to those familiar with the art, can be utilized as excipients for delivery of the therapeutic agent.

In other embodiments, the composition of the invention comprises one or more strains of probiotic bacteria, for example, Lactobacillus rhamnosus HN001 derivative. Again, methods to produce such compositions are well-known in the art, and may utilise standard microbiological and pharmaceutical practices.

It will be appreciated that a broad range of additives or carriers may be included in such compositions, for example to improve or preserve bacterial viability. For example, additives such as surfactants, wetters, humectants, stickers, dispersal agents, stablisers, penetrants, and so-called stressing additives to improve bacterial cell vigor, growth, replication and survivability (such as potassium chloride, glycerol, sodium chloride and glucose), as well as cryoprotectants such as maltodextrin, may be included. Additives may also include compositions which assist in maintaining microorganism viability in long term storage, for example unrefined corn oil, or “invert” emulsions containing a mixture of oils and waxes on the outside and water, sodium alginate and bacteria on the inside.

The composition may comprise a carbohydrate source, such as a disaccharide including, for example, sucrose, fructose, glucose, or dextrose. Preferably the carbohydrate source is one able to be aerobically or anaerobically utilised by probiotic bacteria.

In certain embodiments, the composition comprises a probiotic and a prebiotic, for example fructooligosaccharides, galactooligosaccharides, human milk oligosaccharides and combinations thereof.

It will be appreciated that preferred compositions are formulated to provide an efficacious dose of one or more polar lipids in a convenient form and amount. In certain embodiments, such as but not limited to those where periodic dose need not vary with body weight or other characteristics of the subject, the composition may formulated for unit dosage. It should be appreciated that administration may include a single daily dose or administration of a number of discrete divided doses as may be appropriate. For example, an efficacious dose of one or more polar lipids may be formulated into a capsule for oral administration. In another embodiment an efficacious dose of one or more polar lipids may be provided in one or more serves of a powdered formula reconstituted in water or other liquid, as described in the Example below.

However, by way of general example, the inventors contemplate administration of from about 1 mg to about 1000 mg polar lipids per kg body weight of a composition useful herein per day, preferably about 50 to about 500 mg per kg per day, alternatively about 150 to about 410 mg/kg/day or about 110 to about 310 mg/kg/day. In various embodiments, the inventors contemplate administration of from about 0.05 mg to about 250 mg polar lipids per kg body weight of a composition useful herein.

Examples of fortified drinks are presented herein. Compositions such as these may be formulated so that the concentration of the one or more polar lipids present in the composition is such that an efficacious dose can be prepared using a readily measurable amount of the composition. For example, in certain embodiments, such as for example where the composition is in the form of a powder to be reconstituted in water, the one or more polar lipids is provided at a concentration sufficient to supply an efficacious dose in an amount of formula capable of being easily measured when preparing the formula for administration, such as, for example, with a measured scoop or similar as are commonly provided with powdered formulas.

The compositions useful herein may be used alone or in combination with one or more other therapeutic agents. The therapeutic agent may be a food, drink, food additive, drink additive, food component, drink component, dietary supplement, nutritional composition, medical food, nutraceutical, medicament or pharmaceutical. In various embodiments the therapeutic agent is a meal or meal replacement, for example, an Ensure° shake.

When used in combination with another therapeutic agent, the administration of a composition useful herein and the other therapeutic agent may be simultaneous or sequential. Simultaneous administration includes the administration of a single dosage form that comprises all components or the administration of separate dosage forms at substantially the same time. Sequential administration includes administration according to different schedules, preferably so that there is an overlap in the periods during which the composition useful herein and other therapeutic agents are provided.

Suitable agents with which the compositions useful herein can be separately, simultaneously or sequentially administered include one or more probiotic agents, one or more prebiotic agents, other suitable agents known in the art, and combinations thereof. Useful prebiotics include galactooligosaccharides (GOS), short chain GOS, long chain GOS, fructooligosaccharides (FOS), human milk oligosaccharides (HMO), short chain FOS, long chain FOS, inulin, galactans, fructans, lactulose, peptides, including peptide hydrolysates, and any mixture of any two or more thereof. Some prebiotics are reviewed by Boehm G and Moro G (Structural and Functional Aspects of Prebiotics Used in Infant Nutrition, J. Nutr. (2008) 138(9): 1818S-1828S), incorporated herein by reference. Other useful agents may include dietary fibre such as a fully or partially insoluble or indigestible dietary fibre.

In various embodiments, a composition useful herein includes or is administered simultaneously or sequentially with milk components such as whey protein, whey protein fractions (including acidic or basic whey protein fractions or a combination thereof), glycomacropeptide, lactoferrin, iron-lactoferrin, a functional lactoferrin variant, a functional lactoferrin fragment, a vitamin D or calcium, or combinations thereof. Useful milk component-containing compositions include compositions such as a food, drink, food additive, drink additive, dietary supplement, nutritional composition, medical food or nutraceutical. Milk fractions enriched for these components may also be employed. Useful lactoferrins, fragments and compositions are described in international patent applications WO 03/082921 and WO 2007/043900, both incorporated herein by reference in their entirety.

It should be understood that the additional therapeutic agents listed above (both food based and pharmaceutical agents) may also be employed in a method according to the invention where they are administered separately, simultaneously or sequentially with a composition useful herein.

In various embodiments a composition useful herein may comprise a pharmaceutically acceptable carrier. In various embodiments the composition may be or may be formulated as a food, drink, food additive, drink additive, dietary supplement, nutritional composition, medical food, enteral feeding product, parenteral feeding product, meal replacement, cosmeceutical, nutraceutical, medicament, or pharmaceutical. In one embodiment the composition is in the form of a tablet, a caplet, a pill, a hard or soft capsule or a lozenge. In one embodiment the composition is in the form of a cachet, a powder, a dispensable powder, granules, a suspension, an elixir, a liquid, or any other form that can be added to food or drink, including for example water, milk or fruit juice. In one embodiment the composition further comprises one or more constituents (such as antioxidants) which prevent or reduce degradation of the composition during storage or after administration. These compositions may include any edible consumer product which is able to carry bacteria or bacterial derivatives, including heat-killed, pressure-killed, lysed, UV- or light-treated, irradiated, fractionated or otherwise killed or attenuated bacteria. Examples of suitable edible consumer products include aqueous products, baked goods, confectionery products including chocolate and chewable confectionery such as gums, gels, ice creams, reconstituted fruit products, snack bars, food bars, muesli bars, spreads, sauces, dips, dairy products including yoghurts and cheeses, drinks including dairy and non-dairy based drinks, milk, milk powders, sports supplements including dairy and non-dairy based sports supplements, fruit juice, food additives such as protein sprinkles, dietary supplement products including daily supplement tablets, weaning foods and yoghurts, and formulas, in powder or liquid form. Suitable nutraceutical compositions useful herein may be provided in similar forms.

### 3. Maintaining or Increasing Mobility and/or Vitality

The phrase “maintaining or increasing mobility” as used herein includes within its scope the maintenance or improvement of physical, physiological and/or functional parameters in a subject that affect the ability of the subject to move freely and easily. The phrase includes within its scope, but is not limited to:
a) treating or preventing sarcopenia or one or more sequelae of sarcopenia,
b) maintaining or increasing physical performance,
c) maintaining or reducing net bone loss and/or treating or preventing a condition associated with net bone loss, and/or
d) maintaining or increasing body tone.

The phrase “treating or preventing sarcopenia or one or more sequelae of sarcopenia” and grammatical equivalents as used herein contemplates degenerative loss of skeletal muscle mass and/or muscle strength in a subject, for example, the loss of muscle mass and/or strength associated with aging. In some embodiments the sarcopenia is sarcopenia in a subject aged from about 30, 35, 40 or 45 years of age. In some embodiments the sarcopenia may be present in a subject who is otherwise healthy. In some embodiments the sarcopenia is sarcopenia associated with a sedentary lifestyle. This phrase contemplates the treatment or prevention of sequelae of sarcopenia including (but not limited to): reduced muscle protein synthesis, reduced muscle mass, increased loss of muscle mass, reduced muscle power, strength and/or function, decreased body tone (including increased body fat mass), decreased physical performance (including decreased balance, flexibility, aerobic fitness), increased risk of bone fragility and osteoporosis, increased risk of falls and fractures, and/or decreased physical activity and independence.

In various embodiments treating or preventing sarcopenia comprises
a) maintaining or increasing muscle power,
b) maintaining or increasing muscle strength,
c) maintaining or increasing muscle function,
d) maintaining or increasing lean muscle mass,
e) maintaining or increasing muscle cross-sectional area,
f) maintaining or increasing muscle density, or
g) a combination of any two or more of (a) to (f).

The phrase “maintaining or increasing physical performance” as used herein includes within its scope the maintenance or improvement of musculoskeletal condition and/or function, including (but not limited to) parameters such as
h) maintaining or increasing muscle power,
i) maintaining or increasing muscle strength,
j) maintaining or increasing muscle function,
k) maintaining or increasing balance,
1) maintaining or increasing flexibility,
m) maintaining or increasing aerobic fitness,
n) maintaining or increasing aerobic endurance, and/or
o) maintaining or increasing sports performance.

The phrase “maintaining or reducing net bone loss” as used herein includes within its scope change in physiological and/or physical parameters that affect the rate at which bone mass is reduced. Such parameters may include (but are not limited to):
a) maintaining or increasing bone mineral content,
b) maintaining or increasing bone mineral density,
c) maintaining or increasing bone mass,
d) maintaining or reducing bone turnover,
e) maintaining or reducing bone resorption, or
f) any combination of any two or more thereof.

In various embodiments the condition associated with net bone loss is a skeletal disorder. In various embodiments the condition associated with net bone loss may be selected from the group comprising bone fracture, bone damage following surgery, osteoporosis, rheumatoid arthritis, osteoarthritis, hepatic osteodystrophy, osteomalacia, rickets, osteitis fibrosa cystica, renal osteodystrophy, osteosclerosis, osteopenia, fibrogenesis -imperfecta ossium, secondary hyperparathyrodism, hypoparathyroidism, hyperparathyroidism, chronic renal disease, sarcoidosis, glucocorticoid-induced osteoporosis, idiopathic hypercalcemia, Paget’s disease, osteogenesis imperfecta and oral bone erosion (such as peritonitis or osteonecrosis of the jaw, particularly of alveolar bone). In one embodiment the condition is osteoporosis, osteoarthritis or oral bone erosion. In another embodiment the condition is osteoporosis.

The phrase “maintaining or reducing bone turnover” and its grammatical equivalents and derivatives, refers to changes in the rate of bone formation and/or bone resorption, which results in a net loss or maintenance of bone turnover or remodelling. In various embodiments, bone formation is maintained, increased or decreased. In various embodiments, bone resorption is maintained or decreased. In some embodiments bone turnover is determined by measuring the concentration of bone formation and resorption markers in a subject. In some embodiments the bone formation marker may be P1NP (procollagen type 1 N-terminal propeptide). In some embodiments bone resorption marker may be β-CTx (carboxy-terminal crosslinking telopeptide of type I collagen). Other compounds that directly or indirectly affect bone formation or resorption may also be measured to assess bone turnover, for example, serum parathyroid hormone (PTH) or calcifediol (also referred to as 25-hydroxyvitamin D or 25(OH)D).

The phrase “maintaining or increasing body tone” as used herein includes maintaining or increasing the lean muscle mass of a subject or maintaining or reducing body fat mass of a subject as measured across the whole body, or a region of the body, for example, the arms, legs or trunk. The phrase also includes maintaining or increasing muscle cross-sectional area or density as measured across the whole body or a region of the body, for example, the femur (mid-thigh) or tibia (calf).

The term “vitality” as used herein generally refers to a subject’s positive feeling of aliveness and energy as defined by Ryan and Frederick (1997; Journal of Personality, 65, 529-565). Vitality is associated with a subject’s perceived feelings of factors including alertness, robustness, energy, good health, strength and stamina. Increased vitality is associated with increased daily physical activity and decreased sedentary time.

In one embodiment, vitality may be assessed by determining the score of subject on the subjective vitality scale. The subjective vitality scale refers to the Subjective Vitality Scale - State Level version as defined in Bostic et al., Social Indicators Res. 2000;52:313-24. In one embodiment a subject is considered to have maintained or increased vitality if their score on the subjective vitality scale is unchanged or has increased by at least about 2, 2.25, 2.5, 2.6, 2.75, 3, or at least about 3.25.

The phrase “sedentary” as used herein refers to time when a subject is substantially motionless, for example, when the subject is sitting or lying (but not sleeping). In some embodiments, a subject is sedentary when an accelerometer worn by the subject measures 250 counts per minute or less.

An accelerometer worn by the subject may also be used to measure the intensity of physical activity or exercise. Any suitable accelerometer, typically worn by the subject at the level of the iliac crest on the hip, known in the art may be used. In various embodiments
a) light physical activity may comprise activity that generates from about 250 to about 1951 counts per minute,
b) moderate physical activity may comprise activity that generates from about 1952 to about 5724 counts per minute, and
c) vigorous physical activity may comprise activity that generates greater than about 5725 counts per minute
as measured by an accelerometer worn on the subject’s hip.

It will be appreciated that different compositions of the invention may be formulated with a view to administration to a particular subject group. For example, the formulation of a composition suitable to be administered prophylactically may differ to that of a composition formulated for administration once symptoms of impaired mobility and/or vitality are present in the subject.

In some embodiments the method comprises administering the composition to a pregnant subject. In this embodiment the composition may comprise a maternal formula or maternal supplement.

The compositions useful herein may be used alone. In a preferred embodiment the compositions are administered in combination with exercise. In one embodiment the exercise may comprise one or more exercise sessions per week comprising progressive resistance training (PRT), aerobic exercise, and/or balance and flexibility training. In various embodiments the subject undertakes exercise on at least one, 2, 3, 4 or 5 days per week. In various embodiments, the exercise is from about 20 minutes to about 2 hours in duration.

The method of achieving the above effects comprises the step of administering to a subject in need thereof an effective amount of a composition, as described herein, according to methods as described herein. It should be understood that in some embodiments consumption of a composition of the invention by a subject may result in at least maintenance of one of the above effects. For example, maintaining lean muscle mass, or maintaining net bone loss.

The efficacy of a composition useful according to the invention can be evaluated both in vitro and in vivo, and methods for such are known in the art, including those described in the examples below. Briefly, in one embodiment a candidate composition can be tested for its ability, to for example, maintain or increase bone mineral density or maintain or increase muscle power, function, or strength or maintain or increase lean muscle mass. In some embodiments, for in vivo studies, a composition can be fed to or administered to an animal (e.g., a mouse) and its effects on bone mineral density then assessed. Based on the results, an appropriate dosage range and administration route can be determined.

Suitable methods of assessing and measuring muscle power, strength, and function, balance, flexibility, aerobic fitness and endurance, net bone loss and body tone are described in detail in the examples.

Muscle power may be assessed using measures known in the art including the Leonardo Mechanography 5 step stair climbing power, 10 step stair climbing power, the sit-to-stand test and/or vertical jump test using standard test methods such as those provided in the example. Measures including static squat jump height, static squat jump power, countermovement jump height, countermovement jump power, fast ascent peak power, regular pace ascent power, and concentric power may be determined in order to assess muscle power.

In one embodiment static squat jump height may be determined by measuring the height of a static jump performed by the subject. In one embodiment static squat jump power-to-weight ratio may be determined by measuring the power of a static jump performed by the subject and calculating power per kilogram body mass of the subject. In one embodiment a static squat jump is a jump wherein the subject bends their knees and hips to a self-selected depth to adopt a bending position, holds this position at least momentarily, and jumps as high as possible while keeping their hands on their waist.

In one embodiment countermovement jump height may be determined by measuring the height of a countermovement jump performed by the subject. In one embodiment countermovement jump power-to-weight ratio may be determined by measuring the power of a countermovement jump performed by the subject and calculating power per kilogram body weight of the subject. In one embodiment a countermovement jump is a jump wherein the subject bends their knees and hips to a self-selected depth to adopt a bending position, and immediately jumps as high as possible while keeping their hands on their waist.

In various embodiments static squat jump power or countermovement jump power may be measured using a suitable force plate on which the subject performs the jump, for example, the Advanced Mechanical Technology, Inc. (AMTI) Accugait ACG force plate.

In one embodiment fast ascent peak power may be determined by having the subject perform a 10 step stair climbing test at fast pace and calculating ascent power using the equation: Power (watts) = 9.81 × body mass (kg) × vertical height (m) × number of steps / time (sec). In one embodiment regular pace ascent power-to-weight ratio may be determined by having the subject perform a five step stair testand calculating ascent power per kilogram body weight of the subject. In one embodiment, concentric power-to-weight ratio is determined by measuring concentric power while the subject performs a five consecutive sit-to-stand test and calculating power per kilogram body weight of the subject.

In one embodiment the 10 step stair climbing test may comprise the subject ascending a flight of 10 stairs as fast as possible without running. In one embodiment the five step stair test may comprise ascending a flight of five stairs having a step height of 17.5 cm and a step depth of 28 cm as fast as possible without running. An example of such a test is the Leonardo Mechanography Stair A Test (Adult version; Novotec Medical, Pforzheim, Germany). Power may be measured using one or more sensors, optionally integrated into the stairs.

In one embodiment the five consecutive sit-to-stand test may comprise the subject being seated in a chair with arms folded across the subject’s chest and standing fully upright then returning to a seated position as quickly as possible performed five times consecutively. In one embodiment concentric velocity is measured using an accelerometer fitted to the subject’s hip.

Muscle strength may be assessed using any measure known in the art, for example, grip strength, dorsiflexion strength and/or leg press strength, such as one-repetition maximum leg press strength.

In one embodiment grip strength may be maximal isometric grip strength. In one embodiment maximal isometric grip strength may be determined by having the subject adopt a sitting position with shoulder adducted and neutrally rotated, elbow flexed at an angle of 90 degrees, forearm neutral and hand slightly extended and then squeeze the handle of a dynamometer with the hand using maximal effort and measuring maximal grip strength. Grip strength may be measured in one or both hands. Suitable hand-held dynamometers are well known in the art.

In one embodiment dorsiflexion strength may be maximal isometric dorsiflexion strength. In one embodiment maximal isometric dorsiflexion strength determined by having the subject adopt a sitting position on a 45 cm high chair with one foot strapped securely to a spring-gauged plate attached to a strain gauge load cell of a dynamometer and then dorsiflex the leg using maximal effort and measuring maximal dorsiflexion strength. Dorsiflexion strength may be measured in one or both legs. Suitable dorsiflexion dynamometers are well known in the art.

In one embodiment one-repetition maximum leg press strength may be determined by measuring the maximum load that can be lifted through a full range of motion by the subject on a bilateral leg press while maintaining correct leg press technique throughout. Correct leg press technique includes keeping the knees in line with the toes. Other elements of correct technique are known in the art.

Muscle function, for example, neuromuscular function may be assessed using measures known in the art including for example, a four metre walk test to assess gait speed, a timed up and go test, a four-square step test (FSST) or a choice stepping reaction time test described herein in the example.

For example, the choice stepping reaction time test may be used to determine movement time, a useful measure of neuromuscular function. In one embodiment movement time may be determined by having the subject perform a choice stepping reaction time test. In one embodiment the choice stepping reaction time test may comprise having the subject stand on a choice reaction mat comprising six rectangular panels, each panel being 32 ×13 cm, illuminating one panel at a time in a random order and having the subject step on to each illuminated panel as quickly as possible and measuring the time from movement initiation to foot contact with the panel. A mean movement time of multiple repetitions may be measured.

Balance may be assessed using any suitable measure known in the art, for example, a single leg standing balance test with eyes open or closed. In one embodiment balance may be determined by having the subject balance barefoot on one foot with eyes open or closed and measuring the length of time the subject is able to maintain their non-weight bearing foot raised to ankle height. A mean time of multiple attempts may be measured.

Flexibility may be assessed using any suitable measure known in the art, for example, a sit-and-reach test, which assesses flexibility of the lower back and hamstrings or maximal ankle joint range of motion. In one embodiment maximum stretch distance is determined by having the subject adopt a sitting position on a floor with legs extended and then stretch forward over the subject’s feet with the arms fully extended and hands overlapping and measuring the maximum distance that the subject is able to maintain for at least about 3 seconds from the feet to the tip of the middle finger.

Aerobic fitness may be assessed using any suitable measure known in the art, for example, mean step test number as determined using a step test. In one embodiment mean step test number is determined by having the subject adopt a standing position and raise each knee repeatedly to a string positioned at a height midway between the knee cap and the front of the iliac crest at maximal speed and measuring the number of steps completed in two minutes.

Net bone loss may be assessed using any suitable measure known in the art. In one embodiment total body mineral content is determined by dual energy x-ray absorptiometry (DXA). In various embodiments serum parathyroid hormone (PTH), serum 25-hydroyxvitamin D, plasma carboxy-terminal crosslinking telopeptide of type I collagen (β-CTx) and/or plasma procollagen type 1 N-terminal propeptide (P1NP) concentrations are measured from a blood sample obtained from the subject using any method known in the art, such as the methods described herein in the example. In one embodiment corrected CTX-II (corrCTx-II) is measured in a urine sample obtained from the subject using any method known in the art, for example, the ELISA method described herein in the example.

Body tone may be assessed using any suitable measure known in the art. In various embodiments maintaining or increasing body tone may be assessed by determining total body or regional fat mass, total body or regional lean muscle mass, muscle cross-sectional area or muscle density.

In various embodiments fat mass and/or lean muscle mass may be determined by dual energy x-ray absorptiometry (DXA). In one embodiment total body fat mass and/or lean muscle mass may be determined. In various embodiments regional fat mass and/or lean muscle mass may be determined. In various embodiments the regional fat mass may be appendicular or trunk fat mass. In various embodiments the regional lean muscle mass may be appendicular or trunk lean muscle mass.

In various embodiments muscle cross-sectional area or density may be determined by peripheral quantitative computed tomography (pQCT). In various embodiments the muscle cross-sectional area or density may be femur or tibia muscle cross-sectional area or density. In one embodiment femur cross-sectional area or density may be measured at a site located at 50% of the length of the femur (mid-thigh). In one embodiment tibia cross-sectional area or density may be determined at a site located at 66% of the length of the tibia (where the largest calf muscle diameter is typically located).

Various aspects of the invention will now be illustrated in non-limiting ways by reference to the following example.

### A. Example

The purpose of this example was to investigate the effect of administering compositions comprising polar lipids on mobility and vitality.

### 1. Methods

244 middle aged and habitually sedentary women were recruited. Exclusion criteria included: 1) aged <45 or >65 years; 2) BMI <17 or >40 kg/m²; 3) current or prior participation in resistance exercise (>1 week) and/or moderate-intensity physical activity ≥150 minutes per week in the past 6 months; 4) sedentary time <8 hours per day as determined from the Past-day Adults' Sedentary time (PAST) questionnaire (Clark, et al. Med Sci Sports Exerc. 2013 45(6): 1198207); 5) currently participating in a weight loss program (if weight stable <3 months); 6) dietary calcium intake >900 mg/day as determined from the 17-item 24 hour food recall (Green et al. Asia Pac J Clin Nutr. 2002;11(2): 147-50); 6) regular (>3 times per week) anti-inflammatory use over the past 3 months (including aspirin, ibuprofen, omega 3 supplements/fish oils); 7) use of oral hormone replacement therapy in past 12 months; 8) osteoporosis or any low-trauma fracture in the past 12 months; 9) any other metabolic bone disease or current (or in past 12 months) or use of bisphosphonates; 10) any endocrine-related disorders including diabetes mellitus.

Participants were randomised into one of two treatment groups. Both groups participated in a multi-component exercise programme.
Placebo group: consumption of a placebo drink
Treatment group: consumption of a fortified drink

### 1.1 Nutritional drinks

The composition of the fortified drink and placebo drink is shown in Table 1. Daily intake was two serves comprising 28.35 g or 30 g powder for the fortified and placebo drink, respectively, reconstituted in 150 mL water.

Table 1

COMPOSITION OF FORTIFIED DRINK AND PLACEBO DRINK

| | Fortified drink | | Placebo drink | |
|---|---|---|---|---|
| Nutrient | Per 100 g powder | Daily serve (2 × 28.35 g serve) | Per 100 g powder | Daily serve (2 × 30 g serve) |
| Energy, kJ | 1484 | 836 | 1393 | 836 |
| Protein, g | 32 | 18 | 5.3 | 3.2 |
| Fat, g | 2.1 | 1.23 | 5.3 | 3.2 |
| Carbohydrate, g | 51.5 | 29 | 83.3 | 50 |
| Polar lipid (Phospholipids) mg | 700 | 400 | - | - |
| Vitamin B6 mg | 3.2 | 1.8 | - | - |
| Vitamin B12 µg | 2.65 | 1.49 | - | - |
| Vitamin C mg | 47 | 27 | - | - |
| Vitamin D, µg | 27 | 15 | Trace | Trace |
| Vitamin E mg | 7 | 4.05 | - | - |
| Calcium, mg | 2129 | 1200 | 97 | 58 |
| Zinc, mg | 7.2 | 4.0 | - | - |
| Magnesium, mg | 306 | 173 | - | - |
| Collagen (hydrolysed fish), g | 0.33 | 0.2 | - | - |
| ACE K, mg | 0.014 | 0.0084 | - | - |
| Sucralose, mg | 0.00470 | 0.00282 | - | - |
| Flavouring (vanilla) | 0.212 | 0.127 | - | - |

### 1.2 Exercise Programme

All participants were prescribed an individually-tailored and supervised progressive resistance training (PRT), challenging balance and mobility program. Participants trained on two non-consecutive days per week for 16 weeks. Starting in week 5, participants were also asked to complete one home-based training session per week.

Each exercise session lasted approximately 60 minutes, and included a warm-up and cool down, 30-40 minutes of moderate to high intensity PRT and at least two challenging balance, mobility and postural exercises. The home exercise program was designed to be completed in appropriately 20 minutes and consisted of functional exercises aimed at improving muscle strength, balance and flexibility.

### 1.3 Measurements

The following measurements were performed at baseline and 16 weeks, unless otherwise stated.

### Functional Muscle Power

### Leonardo Mechanography Five Step Stair Climbing Power

Stair climbing time and muscle power was measured using the Leonardo Mechanography Stair A Test (Adult version; Novotec Medical, Pforzheim, Germany) and analysed using the Leonardo Mechanography v4.3 RES (Novotec Medical, Pforzheim, Germany) software. Participants completed two familiarization trials for both stair ascent and descent (with a short rest between trials) at their self chosen speed followed by two test trials. Participants then completed the stair ascent and descent as fast as possible without running. Peak stair climbing time (seconds) and power (Watts per kg) were recorded for both the stair ascent and descent.

### 10 Step Stair Climbing Power

Stair climbing time and muscle power was also assessed using the traditional 10 step stair climb test described in Bean et al. Arch Phys Med Rehabil. 2007 May;88(5):604-9. Participants were given one practice trial and two test trials (with a 1-minute rest between trials) for both stair ascent and descent. In both tests the fastest stair ascent and descent time was recorded. Stair climb power (ascent only) was estimated using the following equation: Power (watts) = 9.81 × body mass (kg) × vertical height (m) × number of steps / time (sec).

### Sit-to-Stand Test

Functional lower limb muscle strength and power was assessed using the five consecutive sit-to-stand (STS) test. Participants started from a seated position in a chair, with arms folded across their chest, and were instructed to stand fully upright and return to the seated position five times as fast as possible. Participant completed one practice trial and one test trial with the time to complete the test recorded using a stopwatch. Participants were fitted with a triaxial accelerometer (x-BIMU Bluetooth Kit, x-io Technologies Limited, Ascot, UK, gyroscope ±2,000°/s, accelerometer ±16 g, 16-bit A/D conversion, sampled at 256 Hz) worn at the right hip from which mean concentric velocity (m/sec) and power relative to body weight (Watts per kg) were calculated.

### Vertical Jump Test

Vertical jump muscle power production was assessed from a static squat jump (SSJ) and countermovement jump (CMJ) test while participants were standing on a force plate (Advanced Mechanical Technology, Inc. [AMTI] Accugait [model number: ACG], Watertown, MA, USA). For the SSJ, participants made a downwards movement from the standing position by bending their knees and hips to a self-selected depth and held this position briefly, then jumped as high as possible while keeping their hands on their waist. For the CMJ, participants squatted and then immediately jumped as high as possible. each test, the peak vertical jump height (cm), velocity (m/sec) and power (Watts per kg) were recorded. For both the SSJ and CMJ, three test trials were completed with a 30-second rest between trials.

### Functional Mobility

### Four Metre Walk Test

The 4-metre walk test was used to assess gait speed. Participants were required to walk in a straight line at their normal comfortable walking speed and at their maximum walking speed across 4 metres. Participants began walking 2 metres prior to and continued walking 2 metres past the 4 metre marker to ensure that a measure of usual walking speed was measured. The time to complete each test was assessed using timing gates (Swift Speedlink Performance Equipment systems). Participants were given one practice trial before completing two test trials at each speed. The fastest time (to the nearest millisecond) was recorded.

### Timed Up and Go Test

Participants were seated in a chair (height 45 cm) that was placed at the end of a marked 3-metre walkway and instructed to stand up, walk as quickly and safely as possible for 3 metres, turn back to the chair and sit down. To minimise any ceiling effects and to make the test more challenging, the test was repeated while participants held a cup of water (filled to 0.5 cm from the rim) throughout the test. During this test participants could hold the cup in either hand and were instructed to not spill any water. Under each condition participants were given one practice trial and two test trials. A third trial was performed if the participant spilled any water. A stopwatch was used to record the time taken to complete the test and recorded to 0.1 seconds.

### Four-Square Step Test (FSST)

Participants were instructed to step forward, sideways, and backwards over two canes resting flat on the floor in a cross formation. The test began with the participant moving first in a clockwise direction and returning in a counter-clockwise direction to the start square. Participants were instructed to complete the task as quickly as possible without touching or stepping on the canes and, if possible, to face forward during the entire sequence. They were also instructed to ensure that both feet made contact with the floor in each square. After one practice trial, participants completed the test twice and the time (in seconds) taken to complete each sequence was measured with a stopwatch and recorded as the final score.

### Choice Stepping Reaction Time

Participants stood on a non-slip choice reaction mat (0.8 × 0.8 m) that contains six rectangular panels (32 × 13 cm), two in front and behind and one to the side of each foot (Neuroscience Research Australia, Sydney, Australia). One panel per trial illuminated in a random order, and participants were instructed to step onto corresponding illuminated panel as quickly as possible, using the left foot only for the three left panels (front and side) and the right foot only for the three right panels. After completing a practice trial, participants completed a single trial that involved 12 target stepping actions in which 12 green arrows appeared in a random sequence (single-task condition). The mean stepping response time (in milliseconds) was measured as the time period between the illumination of an arrow and the foot making contact. This was also additionally subdivided into: 1) decision (reaction) time from the illumination of the arrow to movement initiation (lift off) and 2) movement time from movement initiation to foot contact with the arrow/mat (‘step down’).

### Muscle Strength

### Leg Press Muscle Strength, Velocity and Power

Maximal muscle strength was assessed by a one-repetition (1-RM) test on the bilateral leg press using the Keiser pneumatic resistance training machine fitted with A420 electronics. The 1-RM is the maximum load that can be lifted once throughout the full range of motion while maintaining correct technique. Prior to the test, participants completed the two-minute step test as a warm-up. To determine 1-RM, each participant performed a warm-up set of 8 repetitions at a load of approximately 50% of body weight. After the successful completion of a further 5 repetitions at a higher load, the weight was increased incrementally until only one repetition with correct technique could be completed. Participants were given ~1-2 minutes rest between each attempt. Following a 15-minute rest, leg extensor concentric power at 40% and 70% of 1-RM was assessed. Participants were asked to perform five repetitions as fast as possible through the concentric (pushing) phase of each repetition. The highest concentric power, velocity and force from the five repetitions was recorded and used for analysis.

### Ankle Dorsiflexion Muscle Strength

Bi-lateral maximal isometric dorsi-flexion strength was assessed using a dorsi-flexion dynamometer (Neuroscience Research Australia, Sydney, Australia). Participants were instructed to sit on a 45-cm high chair with the foot strapped securely to a spring-gauged plate attached to a strain gauge load cell. Participants were instructed to perform one practice trial followed by two maximal effort muscle contractions, interspersed by a 15-second rest. For analysis, maximal dorsi-flexion strength (kg) of each leg was recorded.

### Grip Strength

Bi-lateral maximal isometric grip strength was assessed using a hand-held dynamometer (Jamar dynamometer, Asimov Engineering Co., Los Angeles, CA, USA). Participants were instructed to sit on a standard height chair with their shoulder adducted and neutrally rotated, elbow flexed at 90°, forearm neutral and hand slightly extended. They were instructed to perform one practice trial, followed by two maximal effort muscle contractions by squeezing the handle of the dynamometer as forcefully as possible. For analysis, maximal grip strength (kg) of each hand was recorded.

### Balance

Single-leg standing balance with eyes open and closed was assessed using an AMTI Accugait force platform. With shoes removed participants were asked to stand on one leg in the centre of the force plate, with their non-weight bearing foot raised to ankle height and eyes open for up to 30 seconds. If they were able to hold this position, they were then asked to repeat the test with their opposite leg. If they were unable to hold the position for 30 seconds, the time to fail was recorded. All participants were allowed three attempts. Participants were then asked to repeat the test with their eyes closed, on both legs. For participants that were able to hold the single leg standing position for 30 seconds, centre of pressure (COP) velocity (m/sec) and anterior-posterior (A-P) and medial-lateral (M-L) displacement (in cm) were recorded.

### Flexibility

### Sit-and-Reach Test

Flexibility of the lower back and hamstrings was measured using the sit-and-reach test at baseline, 2 and 4 months. Participants sat on the floor with shoes removed and legs extended and feet flat against the test box (Figure Finder Flex-tester, Novel Products Inc, Rockton, IL, USA). Participants were instructed to reach forward with their overlapping hands (third digits overlapping) as far as possible. The maximum stretch held for at least 3 seconds was recorded. During each attempt, the participant was instructed to exhale and drop their head forward whilst reaching. Three trials were completed with the greatest reach (in cm) taken for analysis.

### Maximal Ankle Joint Range of Motion

Maximal ankle dorsiflexion range of motion was measured using the weight-bearing lunge test as described by Konor et al. Int J Sports Phys Ther. 2012 Jun;7(3):279-87. Maximal dorsiflexion ROM was defined as the maximum distance of the toe from the wall while maintaining contact between the wall and knee without lifting the heel. Participants were given a practice trial per limb and completed two test trials per limb, with the highest score for each side used.

### Aerobic Fitness

Participants were asked to step as many times as possible in place (not running) for 2 minutes, raising both knees to a predetermined (standardized) height marked by a string which was secured at a level midway between the knee cap and the front of the iliac crest (as described in Rikli and Jones, Senior Fitness Test: Champaign, IL: Human Kinetics, 2001). The number of times the right knee touched the string was recorded (i.e. completed one full step). If the proper knee height could not be maintained the participant was asked to slow down or to stop until they regained the proper form, but the time kept running.

### Biochemical Measurements of Bone Health

Fasted, resting morning venous blood samples were collected at baseline and 4 months. Serum 25-hydroxyvitamin D was assessed at baseline and 4 months using a validated liquid chromatography/mass spectrometry/mass spectrometry (LC/MS/MS) method on an AbSciex Triple Quad 5500 LC/MS/MS system with an inter-assay %CV of 3.8-8.3%. Serum intact parathyroid hormone (PTH) was assessed using a commercial chemiluminescent immunoassay (Access/DXI PTH assay, Beckman Coulter, Inc. Fullerton, CA, USA) performed on a Beckman Coulter Unicel DXI 800. The inter-assay %CV ranged from 6.6-8.3%. Plasma carboxy-terminal crosslinking telopeptide of type I collagen (β-CTX), as a marker of bone resorption, was determined using an Elecsys (β-Cross Laps electrochemiluminescence immunoassay (Roche Diagnostics, IN, USA), with an inter-assay %CV of 3.3-4.5%. Plasma procollagen type 1 amino-terminal peptide (P1NP), as a marker of bone formation, was determined using an Elecsys total PINP electrochemiluminescence immunoassay (Roche Diagnostics, IN, USA), with an inter-assay %CV of 2.3-5.3%.

Urine creatinine was measured by the standard Jaffe method and this was used to calculate corrected CTX-II.

### Body Composition

Dual energy x-ray absorptiometry (DXA) was used to assess total body and regional (arms, legs and trunk) lean tissue mass, fat mass and percentage body fat and total body bone mineral content (BMC) (Lunar iDXA, GE Medical Systems, Madison WI, Encore version 16).

Appendicular lean mass (ALM) was calculated from the sum of lean tissue mass in both the right and left arms and legs derived from the total body scan. The short-term co-efficient of variation (CV) for repeated measurements of total body lean mass and fat mass in our laboratory ranges from 1.0-1.7%.

Muscle cross-sectional area (CSA) and muscle density, as a surrogate measure of muscle adiposity, at the 50% femur and 66% tibia site were assessed using peripheral quantitative computed tomography (pQCT) (XCT 3000, Stratec Medizintechnik GmbH, Pforzheim, Germany). After performing a scout view of the distal end of the femur and tibia, scans were placed at the 50% femur and 66% tibia site. Slice thickness was 2.3 mm, and voxel size was set at 0.3 mm with a scanning speed of 10 mm/s. Subcutaneous fat CSA was determined by selecting the area with thresholds -40 to +40 mg/cm³ HA density and muscle CSA was determined by subtracting the total bone CSA (threshold, 280 mg/cm³) and subcutaneous fat CSA from the total area of the 50% femur or 66% tibia (threshold, -40 mg/cm³). The CV for femur muscle CSA in the lab was 1.3 %.

### Physical Activity

Daily sedentary time, and light and moderate to vigorous habitual physical activity were assessed at baseline, 2 and 4 months over a 7-day period using an accelerometer sampling at 30 Hz attached to a belt at the waist (level of iliac crest on the right hip) using standard procedures (ActiGraph wGT3X-BT, ActiGraph LLC, USA). The accelerometers measured accelerations at the hip in counts per minute to determine the average time spent each day at different intensities which was defined as follows: sedentary (≤250 counts/min), light (251-1951 counts/min), moderate (1952-5724 counts/min), and vigorous (≥5725 counts/min) intensity activity. Step count and time spent sitting and standing was estimated using an inclinometer (activPAL3 attached to the participant’s thigh).

### Vitality

Subjective vitality was assessed using the 6-item version of the Subjective Vitality Scale - State Level version (Bostic et al., Social Indicators Res. 2000;52:313-24.). The response to each question was summed to generate a total score ranging from 6 to 42.

The 11-item Chalder Fatigue Scale (CFS) was used as a measure of self -reported fatigue severity over the last month as well as physical and mental fatigue (Cella and Chalder, J Psychosom Res. 2010 Jul;69(1):17-22). Participants rated each question on a 4-point Likert scale (0=less than usual; 1=no more than usual; 2=more than usual; 3=much more than usual) with the score summed to provide a total score ranging from 0 to 33. High scores represent high levels of fatigue. The subscales of physical and mental fatigue were determined by summing items 1-7 and 8-11, respectively.

### Statistical Analysis

All statistical analyses were conducted using Stata Statistical Software, release 15.0 (Stata, College Station, TX). All data was checked for outliers and normality, with the residual of any non-normally distributed data checked prior to analysis. Within group changes in the outcome measures were expressed as either the absolute or percentage change from baseline. Between-group differences were calculated by subtracting the within-group changes from baseline for the fortified milk group from the within-group changes for the placebo group after 2 and 4 months when appropriate. Generalized linear mixed models with random effects were used to analyse differences between the groups after 2 and 4 months when appropriate. For data that was non-normality distributed generalized linear models with a gamma distribution were used. For presentation of non-normally distributed data, percentage change was calculated based on log transformed data using the absolute difference from baseline in the log-transformed data multiplied by 100. All data were analysed by using an intention-to-treat approach after which sensitivity analysis (per protocol) was performed by only including women with ≥ 66% adherence to the exercise program and ≥ 90% adherence to the supplements. In comparing changes between groups, the results were analysed unadjusted and adjusted for moderate vigorous physical activity. Analysis of covariance (ANCOVA) with the change scores as the outcome, group as the fixed factor and baseline values as the covariates were also performed. For blood pressure and lipid measures, use of anti-hypertensive medication or lipid-lowering medication were included as covariates. All baseline data are presented as means ± SD or median and interquartile range and all change data are reported as means with 95% CI unless stated. Significance was set at P<0.05.

### Results

108 women in each treatment group completed the programme and were assessed at 4 months. Changes in the various measures of mobility and vitality over the four months are presented below.

### 2.1 Functional Muscle Power

### Stair Climbing Time and Muscle Power

Results are shown in Table 2.

Table 2

STAIR CLIMBING ASCENT AND DESCENT TIME AND POWER AT REGULAR (NORMAL) PACE AND FAST PACE

| Characteristics | n | Fortified drink | n | Placebo drink | Between-Group Difference (95% Cl) |
|---|---|---|---|---|---|
| 10 stair Test | | | | | |
| Fast Ascent Time | | | | | |
| Baseline, sec | 123 | 3.46 ± 0.50 | 121 | 3.43 ± 0.45 | |
| Δ 4 months | 107 | -0.11 (-0.16, -0.06) † | 108 | -0.08 (-0.13, -0.03) † | -0.03 (-0.10, 0.04) |
| Fast Ascent Peak Power | | | | | |
| Baseline, W | 123 | 393 ± 90 | 121 | 392 ± 85 | - |
| % Δ 4 months | 107 | 4.0 (2.5, 5.6) † | 108 | 3.6 (2.0, 5.2) † | 0.4 (-1.8, 2.7) |
| Fast Descent Time | | | | | |
| Baseline, sec | 123 | 3.21 ± 0.59 | 121 | 3.18 ± 0.50 | - |
| Δ 4 months | 107 | -0.09 (-0.16, -0.02) † | 108 | -0.04 (-0.09, 0.02) | -0.06 (-0.14, 0.03) |
| Regular Pace, 5 Stair Test | | | | | |
| Ascent Time | | | | | |
| Baseline, sec | 123 | 4.10 ± 0.39 | 121 | 4.01 ± 0.37 | - |
| Δ 4 months | 108 | -0.09 (-0.15, -0.02) ** | 108 | -0.02 (-0.07, 0.04) | -0.07 (-1.6, 0.01) |
| Ascent Power | | | | | |
| Baseline, W/kg | 123 | 6.22 ± 0.85 | 121 | 6.24 ± 0.84 | - |
| % Δ 4 months | 108 | 1.5 (-0.6, 3.7) | 108 | 0.0 (-1.9, 1.8) | 1.5 (-1.3, 4.4) |
| Descent Time | | | | | |
| Baseline, sec | 123 | 4.00 ± 0.42 | 121 | 3.93 ± 0.42 | - |
| Δ 4 months | 108 | -0.13 (-0.19, -0.07) † | 108 | -0.04 (-0.10, 0.03) | -0.09 (-0.18, 0.00) |
| Descent Power | | | | | |
| Baseline, W/kg | 123 | 7.04 ± 1.25 | 121 | 6.83 ± 1.33 | - |
| % Δ 4 months | 108 | 3.8 (0.0, 7.6) | 108 | 4.2 (1.7, 6.7) ** | -0.4 (-4.9, 4.1) |
| Fast Pace, 5 Stair Test | | | | | |
| Ascent Time | | | | | |
| Baseline, sec | 123 | 3.35 ± 0.36 | 121 | 3.35 ± 0.32 | - |
| Δ 4 months | 108 | -0.08 (-0.13, -0.03) † | 108 | -0.07 (-0.12, -0.02) ** * | -0.01 (-0.08, 0.05) |
| Ascent Power | | | | | |
| Baseline, W/kg | 123 | 7.38 ± 1.22 | 121 | 7.43 ± 1.28 | - |
| % Δ 4 months | 108 | 3.9 (1.2, 6.7) | 108 | 4.3 (1.0, 7.6) * | -0.4 (-4.6, 3.9) |
| Descent Time | | | | | |
| Baseline, sec | 123 | 3.26 ± 0.36 | 121 | 3.23 ± 0.34 | - |
| Δ 4 months | 108 | -0.08 (-0.14, -0.03) † | 108 | -0.03 (-0.07, 0.01) | -0.05 (-0.12, 0.01) |
| Descent Power | | | | | |
| Baseline, W/kg | 123 | 9.24 ± 2.51 | 121 | 9.13 ± 2.07 | - |
| % Δ 4 months | 108 | 4.4 (0.1, 8.7) | 108 | 3.9 (0.3, 7.5) | 0.5 (-5.1, 6.1) |

Baseline values are means ± SD and within and between group differences are unadjusted absolute or percentage changes with 95% Cl. W, Watts. Net differences (95% Cl)were calculated by subtracting within group changes rom baseline for the fortified drink group from the within group changes in the placebo group. * P<0.05, ** P<0.01, † P<0.001 within-group change relative to baseline; # P=0.06; P<0.05 between-group difference for the change relative to baseline (group-by-time interaction).

### Vertical Jump Muscle Power

Results are shown in Table 3.

Table 3

STATIC SQUAT JUMP (SSJ) AND COUNTERMOVEMENT JUMP (CMJ) HEIGHT AND POWER

| Characteristics | n | Fortified drink | n | Placebo drink | Between-Group Difference (95% Cl) |
|---|---|---|---|---|---|
| SSJ, Height | | | | | |
| Baseline, cm | 123 | 15.7 ± 3.4 | 120 | 15.4 ± 3.2 | - |
| Δ 4 months | 107 | 1.1 (0.8, 1.4) † | 102 | 0.8 (0.4, 1.1) † | 0.4 (-0.1, 0.8) ^{#} |
| SSJ, Power | | | | | |
| Baseline, W/kg | 123 | 14.4 ± 3.5 | 120 | 13.8 ± 3.0 | - |
| % Δ 4 months | 107 | 7.2 (4.7, 9.8) † | 102 | 4.3 (2.3, 6.4) † | 2.9 (-0.4, 6.1) |
| CMJ, Height | | | | | |
| Baseline, cm | 121 | 16.4 ± 3.5 | 119 | 16.1 ± 3.2 | - |
| Δ 4 months | 105 | 1.1 (0.8, 1.5) † | 101 | 0.6 (0.3, 0.9) † | 0.5 (0.04, 1.0) |
| CMJ, Power | | | | | |
| Baseline, W/kg | 121 | 14.8 ± 3.0 | 118 | 14.4 ± 3.3 | - |
| % Δ 4 months | 105 | 7.3 (4.8, 9.8) † | 100 | 6.7 (3.9, 9.5) † | 0.6 (-3.2, 4.3) |

Baseline values are means ± SD and within and between group differences are unadjusted absolute or percentage changes with 95% Cl. Net differences (95% Cl)were calculated by subtracting within group changes from baseline for the fortified drink group from the within group changes in the placebo group. † P≤ 0.001 within-group change relative to baseline; ^{#} P=0.06, P<0.05 between-group difference for the change relative to baseline (group-by-time interaction).

### Five Sit-to-Stand Test (STS)

Results are shown in Table 4.

Table 4

SIT-TO-STAND (STS) TIME, CONCENTRIC VELOCITY AND POWER

| Characteristics | n | Fortified drink | n | Placebo | Between-Group Difference (95% Cl) |
|---|---|---|---|---|---|
| STS Total Time | | | | | |
| Baseline, sec | 123 | 9.92 ± 1.55 | 121 | 9.71 ± 1.36 | - |
| Δ 2 months | 111 | -0.30 (-0.54, -0.07) ** | 110 | -0.15 (-0.37, 0.07) | -0.15 (-0.47, 0.16) |
| Δ 4 months | 108 | -0.95 (-1.18, -0.72) † | 108 | -0.69 (-0.90, -0.48) † | -0.26 (-0.57, 0.05) |
| STS Concentric Velocity | | | | | |
| Baseline, m/sec | 123 | 0.60 ± 0.08 | 120 | 0.60 ± 0.09 | - |
| % Δ 2 months | 112 | 6.4 (4.3, 8.6) † | 109 | 6.6 (4.7, 8.5) † | -0.2 (-3.0, 2.7) |
| % Δ 4 months | 108 | 4.0 (2.2, 5.8) † | 108 | 3.3 (1.8, 4.9) † | 0.7 (-1.7, 3.0) |
| STS Concentric Power | | | | | |
| Baseline, W/kg | 121 | 5.92 ± 0.82 | 121 | 5.88 ± 0.87 | - |
| % Δ 2 months | 112 | 6.7 (4.6, 8.4) † | 109 | 6.5 (4.6, 8.4) † | 0.2 (-2.6, 3.0) |
| % Δ 4 months | 105 | 4.2 (2.5, 5.9) † | 101 | 3.2 (1.7, 4.8) † | 0.9 (-1.4, 3.2) |

Baseline values are means ± SD and within and between group differences are unadjusted absolute or percentage changes with 95% Cl. Net differences (95% Cl)were calculated by subtracting within group changes from baseline for the Red group from the within group changes in the Blue group. † P<0.001 within-group change relative to baseline.

### 2.2 Functional Mobility

Results are shown in Table 5.

Table 5

FOUR METRE GAIT SPEED, TIMED UP-AND-GO AND CHOICE REACTION STEPPING TIME (REACTION AND MOVEMENT TIME) PERFORMANCE

| Characteristics | n | Fortified drink | n | Placebo drink | Between-Group Difference (95% Cl) |
|---|---|---|---|---|---|
| 4-m Gait Speed | | | | | |
| Usual speed | | | | | |
| Baseline, m/sec | 123 | 0.69 ± 0.09 | 121 | 0.68 ± 0.08 | - |
| Δ 4 months | 108 | -0.02 (-0.03, 0.00) * | 108 | 0.00 (-0.01, 0.01) | 0.02 (-0.03, 0.00) |
| Fast speed | | | | | |
| Baseline, m/sec | 123 | 0.50 ± 0.06 | 121 | 0.49 ± 0.06 | - |
| Δ 4 months | 108 | -0.01 (-0.02, 0.00) * | 108 | -0.01 (-0.01, 0.00) * | 0.00 (-0.01, 0.01) |
| Timed-up-and-go | | | | | |
| Self-paced | | | | | |
| Baseline, sec | 123 | 6.07 ± 0.68 | 121 | 5.96 ± 0.63 | - |
| Δ 4 months | 108 | -0.19 (-0.27, -0.11) † | 108 | -0.13 (-0.21, -0.05) † | 0.06 (-0.17, 0.06) |
| Self-paced + manual task | | | | | |
| Baseline, sec | 123 | 8.74 ± 1.44 | 121 | 8.53 ± 1.36 | - |
| Δ 4 months | 108 | -0.19 (-0.41, 0.03) * | 108 | -0.16 (-0.35, 0.02) ^{#} | -0.03 (-0.31, 0.25) |
| Four Square Step Test | | | | | |
| Baseline, sec | 123 | 6.30 ± 0.90 | 121 | 6.24 ± 0.84 | - |
| % Δ 2 months | 111 | --0.35 (-0.50, -0.21 ) † | 110 | --0.32 (-0.46, -0.19) † | -0.03 (-0.23, 0.16) |
| % Δ 4 months | 108 | --0.45 (-0.56, -0.33) † | 108 | --0.45 (-0.57, -0.33) † | -0.00 (-0.16, 0.17) |
| Choice Reaction Stepping Time | | | | | |
| Response Time | | | | | |
| Baseline, msec | 123 | 937 ± 101 | 121 | 940 ± 100 | - |
| Δ 4 months | 108 | -3 (-20, 13) | 107 | -12 (-31, 7) | 9 (-16, 34) |
| Decision/Reaction Time | | | | | |
| Baseline, msec | 123 | 703 ± 71 | 121 | 710 ± 71 | - |
| Δ 4 months | 108 | 12 (-0.2, 25)^{#} | 107 | -10 (-23, 4) | 22 (3, 40) |
| Movement Time | | | | | |
| Baseline, msec | 123 | 234 ± 49 | 121 | 230 ± 48 | - |
| Δ 4 months | 108 | -15 (-23, -6) † | 108 | -4 (-13, 7) | -11 (-24, 2) ^{§} |

Baseline values are means ± SD and within and between group differences are unadjusted absolute or percentage changes with 95% Cl. Net differences (95% Cl)were calculated by subtracting within group changes from baseline for the fortified drink group from the within group changes in the placebo group. * P=0.06, * P<0.05, † P≤ 0.001 within-group change relative to baseline. § P=0.07; P<0.05 between-group difference for the change relative to baseline (group-by-time interaction).

### 2.3 Muscle Strength

Results are shown in Table 6.

Table 6

GRIP, DORSIFLEXION AND ONE-REPETITION MAXIMUM LEG PRESS MUSCLE STRENGTH

| Characteristics | n | Fortified drink | n | Placebo | Between-Group Difference (95% Cl) |
|---|---|---|---|---|---|
| Grip strength, left | | | | | |
| Baseline, kg | 123 | 26.2 ± 4.8 | 121 | 26.0 ± 4.8 | |
| % Δ 4 months | 108 | 4.6 (2.0, 7.2) † | 108 | 2.6 (0.3, 4.8) * | 2.0 (-1.4, 5.5) |
| Grip strength, right | | | | | |
| Baseline, kg | 123 | 28.2 ± 4.9 | 121 | 27.5 ± 5.0 | |
| % Δ 4 months | 108 | 4.2(1.7,6.6)** | 108 | 3.4 (1.2, 5.7) ** | 0.7 (-2.6, 4.1) |
| Dorsiflexion strength, left | | | | | |
| Baseline, kg | 123 | 9.6 ± 2.9 | 121 | 9.5 ± 2.5 | |
| % Δ 4 months | 108 | 8.8 (4.6, 12.9) † | 108 | 5.5 (1.6, 9.4) * | 3.2 (-2.4, 8.9) |
| Dorsiflexion strength, right | | | | | |
| Baseline, kg | 123 | 10.2 ± 3.0 | 121 | 10.0 ± 2.7 | |
| % Δ 4 months | 108 | 4.4 (0.03, 8.8) | 108 | 3.8 (0.2, 7.4) | 0.6 (-5.0, 6.3) |
| Leg press 1-RM | | | | | |
| Baseline, kg | 123 | 171 ± 50 | 121 | 173 ± 51 | |
| % Δ 4 months ¹ | 107 | 24 (21, 27) † | 106 | 22 (18, 24) † | 2 (-1.9, 6.5) |

Baseline values are means ± SD and within and between group differences are unadjusted absolute or percentage changes with 95% Cl. ¹ Change represents the absolute difference from baseline in the log transformed data multiplied by 100. Net differences (95% Cl)were calculated by subtracting within group changes from baseline for the fortified drink group from the within group changes in the placebo group. * P<0.05, ** P<0.01, † P<0.001 within-group change relative to baseline.

### 2.4 Balance

Results are shown in Table 7.

Table 7

SINGLE LEG BALANCE TEST PERFORMANCE WITH EYES CLOSED

| Characteristics | Fortified drink | Placebo drink | Chi-squared (P-value) |
|---|---|---|---|
| SLB Eyes Open 30 seconds - Left \| Right | | | |
| Baseline, passed | 89% \| 90% | 93% \| 93% | |
| Unchanged | 93% \| 93% | 94% \| 97% | |
| Improved | 6% \| 5% | 4% \| 3% | P=0.691 \| 0.276 |
| Deteriorated | 1 % \| 2% | 2% \| 0% | |
| Baseline, passed | 25% \| 23% | 21% \| 25% | |
| Unchanged | 80% \| 76% | 87% 1 80% | P=0.142 \| 0.007 |
| Improved | 16% \| 24% | 7% \| 14% | |
| Deteriorated | 4% \| 0% | 6% \| 6% | |

### 2.5 Flexibility

Results are shown in Table 8.

Table 8

MEAN FLEXIBILITY (SIT AND REACH AND ANKLE JOINT RANGE OF MOTION)

| Characteristics | n | Fortified drink | n | Placebo | Between-Group Difference (95% Cl) |
|---|---|---|---|---|---|
| Sit-and-reach | | | | | |
| Baseline, cm | 123 | 23.1 ± 8.9 | 120 | 25.2 ± 8.7 | |
| Δ 2 months | 107 | 3.4 (2.5, 4.3) † | 107 | 2.5 (1.5, 3.4) † | 1.0 (-0.4, 2.3) |
| Δ 4 months | 108 | 2.7 (2.1, 3.3) † | 108 | 1.3 (0.8, 1.9) † | 1.4 (0.6, 2.2) |
| Ankle joint ROM, left | | | | | |
| Baseline, cm | 123 | 9.55 ± 3.18 | 121 | 9.88 ± 3.27 | |
| Δ 4 months | 108 | 0.47 (0.02, 0.92) | 108 | 0.55 (0.19, 0.90) | -0.08 (-0.64. 0.49) |
| Ankle joint ROM, right | | | | | |
| Baseline, cm | 123 | 9.76 ± 3.33 | 121 | 10.42 ± 3.38 | |
| Δ 4 months | 108 | 0.26 (-0.11, 0.63) | 108 | 0.39 (0.05, 0.72) | -0.13 (-0.62, 0.37) |

Baseline values are means ± SD and within and between group differences are unadjusted absolute or percentage changes with 95% Cl. Net differences (95% Cl)were calculated by subtracting within group changes from baseline for the fortified drink group from the within group changes in the placebo group. * P<0.05, ** P<0.01, † P<0.001 within-group change relative to baseline; P<0.05 between group difference for the change relative to baseline (group-by-time interaction).

### 2.6 Aerobic Fitness

Results are shown in Table 9.

Table 9

MEAN STEP TEST NUMBER IN 2 MINUTES

| Characteristics | n | Fortified drink | n | Placebo drink | Between-Group Difference (95% CI) |
|---|---|---|---|---|---|
| Baseline, number of steps | 47 | 109 ± 17 | 44 | 120 ± 20 | |
| Δ 4 months | 43 | 12 (8, 15) † | 38 | 4 (1, 7) ** | 8 (3, 12) |

Baseline values are means ± SD and within and between group differences are unadjusted absolute or percentage changes with 95% Cl. ROM, range of motion. Net differences (95% Cl) were calculated by subtracting within group changes from baseline for the fortified drink group from the within group changes in the placebo group. ** P<0.01, † P<0.001 within-group change relative to baseline; P<0.01 between-group difference for the change relative to baseline (group-by-time interaction).

### 2.7 Bone Health Markers

Results are shown in Table 10.

Table 10

MEAN PARATHYROID HORMONE (PTH), 25-HYDROXYVITAMIN D (25(OH)D), CARBOXY-TERMINAL CROSS-LINKING TELOPEPTIDE OF TYPE I COLLAGEN (B-CTX), PROCOLLAGEN TYPE 1 AMINO-TERMINAL PEPTIDE (P1NP) AND CORRCTX-II

| Characteristics | n | Fortified drink | n | Placebo | Between-Group Differences (95% Cl) |
|---|---|---|---|---|---|
| PTH | | | | | |
| Baseline, nmol/L | 123 | 4.7 ± 2.2 | 121 | 4.7 ± 1.8 | |
| % Δ 4 months ¹ | 108 | -2.3 (-7.8, 3.3) | 107 | 8.6(3.1,14.1)** | -10.9 (-18.6, -3.1) |
| 25-hydroxyvitamin D | | | | | |
| Baseline, nmol/L | 123 | 61.6 ± 21.4 | 121 | 64.2 ±24.7 | |
| Δ 4 months | 108 | 9.1 (5.7, 12.4) † | 108 | -11.8 (-15.3, -8.2) † | 20.9 (16.0, 25.7) ‡ |
| β-CTx | | | | | |
| Baseline, nmol/L | 123 | 0.53 ± 0.23 | 121 | 0.51 ± 0.22 | |
| % Δ 4 months ¹ | 108 | -16.6 (-22.4, -10.9) † | 106 | -0.2 (-5.6, 5.1) | -16.4 (-24.2, -8.6) ‡ |
| P1NP | | | | | |
| Baseline, nmol/L | 123 | 61.0 ± 25.8 | 121 | 59.1 ± 24.0 | |
| % Δ 4 months ¹ | 108 | -8.9 (-13.0, -4.7) † | 106 | 8.5(2.9,14.0)** | -17.3 (-24.1, -10.5) ‡ |
| corrCTX-ll | | | | | |
| Baseline, nmol/L | 123 | 240 ± 205 | 121 | 239 ± 176 | |
| % Δ 4 months ¹ | 108 | -5.2 (-17.1, 6.6) | 108 | -2.6 (-15.2, 10.1) | -2.7 (-19.9, 14.5) |

Baseline values are medians and IQR or mean ± SD and within and between group differences are unadjusted absolute or percentage changes with 95% Cl. ¹ Change represents the absolute difference from baseline in the log transformed data multiplied by 100. Net differences (95% Cl) were calculated by subtracting within group changes from baseline for the fortified drink group from the within group changes in the placebo group. P<0.01, ‡ P<0.001 between-group difference for the change relative to baseline (group-by-time interaction).

### 2.8 Body Composition

### DXA Body Composition

Results are shown in Table 11.

Table 11

BODY COMPOSITION

| Characteristics | n | Fortified drink | n | Placebo | Between-Group Difference (95% Cl) |
|---|---|---|---|---|---|
| Total body | | | | | |
| Weight Baseline, kg | 123 | 76.5 ± 18.3 | 121 | 75.6 ± 16.2 | |
| Δ 4 months | 108 | 0.3 (-0.01, 0.7) ^{#} | 108 | 0.6 (0.3, 0.9) † | -0.3 (-0.8, 0.2) |
| Total body %fat | | | | | |
| Baseline, % | 123 | 42.5 ± 7.9 | 121 | 42.6 ± 7.1 | |
| Δ 4 months | 107 | -1.1 (-1.3, -0.8) † | 108 | -0.4 (-0.6, -0.2) † | -0.7 (-1.0, -0.3) ‡ |
| Fat mass | | | | | |
| Baseline, kg | 123 | 32.3 ± 12.7 | 121 | 31.7 ± 11.3 | |
| Δ 4 months | 107 | -0.6 (-0.9, -0.3) † | 108 | 0.0 (-0.3, -0.3) | -0.6 (-1.0, -0.2) |
| Lean Mass | | | | | |
| Baseline, kg | 123 | 41.1 ± 6.2 | 121 | 40.7 ± 5.6 | |
| Δ 4 months | 107 | 1.0 (0.8, 1.2) † | 108 | 0.7 (0.5, 0.9) † | 0.3 (0.04, 0.6) |
| Bone mineral content | | | | | |
| Baseline, g | 123 | 2287 ± 326 | 121 | 2293 ± 312 | |
| % Δ 4 months | 107 | 0.2 (-0.05, 0.4) | 108 | -0.2 (-0.4, 0.0) * | 0.4 (0.1, 0.6) |
| Regional | | | | | |
| Arm fat mass | | | | | |
| Baseline, kg | 123 | 3.25 ± 1.24 | 121 | 3.31 ± 1.14 | |
| Δ 4 months | 107 | -0.04 (-0.09, 0.00) * | 108 | -0.01 (-0.04, 0.02) | 0.03 (-0.09, 0.02) |
| Arm lean mass | | | | | |
| Baseline, kg | 123 | 4.15 ± 0.76 | 121 | 4.11 ± 0.66 | |
| Δ 4 months | 107 | 0.12 (0.09, 0.15) † | 108 | 0.13 (0.1, 0.16) † | -0.01 (-0.05, 0.03) |
| Leg fat mass | | | | | |
| Baseline, kg | 123 | 11.24 ± 4.70 | 121 | 11.23 ± 4.30 | |
| Δ 4 months | 107 | -0.09 (-0.20, 0.01) | 108 | 0.07 (-0.04, 0.17) | -0.16 (-0.31, -0.09) |
| Leg lean mass | | | | | |
| Baseline, kg | 123 | 14.60 ± 2.83 | 121 | 14.44 ± 2.46 | |
| Δ 4 months | 107 | 0.37 (0.28, 0.46) † | 108 | 0.33 (0.25, 0.41) † | 0.04 (-0.07, 0.2) |
| Appendicular LM ^{a} | | | | | |
| Baseline kg/m² | 123 | 18.75 ± 3.53 | 121 | 18.55 ± 3.07 | |
| Δ 4 months | 107 | 0.49 (0.39, 0.59) † | 108 | 0.46 (0.36, 0.55) † | 0.03 (-0.1, 0.2) |
| Trunk fat mass | | | | | |
| Baseline, kg | 123 | 16.97 ± 7.53 | 121 | 16.34 ± 6.36 | |
| Δ 4 months | 107 | -0.45 (-0.65, -0.25) † | 108 | -0.05 (-0.21, 0.11) | -0.40 (-0.66, -0.15) |
| Trunk lean mass | | | | | |
| Baseline, kg | 123 | 19.4 ± 2.6 | 121 | 19.2 ± 2.6 | |
| Δ 4 months | 107 | 0.50 (0.36, 0.63) † | 108 | 0.21 (0.07, 0.34) ** | 0.29 (0.10, 0.48) |

Baseline values are means ± SD and within and between group differences are unadjusted absolute or percentage changes with 95% Cl. Net differences (95% Cl) were calculated by subtracting within group changes from baseline for the fortified drink group from the within group changes in the Placebo group. # P<0.06, * P<0.05, ** P<0.01, † P≤ 0.001 within-group change relative to baseline; # P=0.06, P<0.05; P<0.01, ‡ P<0.001 between group difference for the change relative to baseline (group-by-time interaction).

### pQCT Body Composition

Results are shown in Table 12.

Table 12

MEAN PQCT-DERIVED MUSCLE CROSS-SECTIONAL AREA (CSA) AND MUSCLE DENSITY AT THE 50% FEMUR AND 66%

| Characteristics | n | Fortified drink | n | Placebo | Between-Group Difference (95% Cl) |
|---|---|---|---|---|---|
| 50% Femur muscle CSA | | | | | |
| Baseline, cm² | 123 | 89.6 ± 16.5 | 121 | 88.8 ± 13.6 | |
| % Δ 4 months | 107 | 5.0 (4.2, 5.8) † | 108 | 3.2 (2.3, 4.1 ) † | 1.8 (0.6, 2.9) |
| 50% Femur muscle density | | | | | |
| Baseline, mg/cm³ | 123 | 77.5 ± 2.1 | 121 | 77.5 ± 2.2 | |
| % Δ 4 months | 107 | 0.7 (0.3, 1.2) ** | 108 | 0.4 (-0.03, 0.8) | 0.4 (-0.2, 1.0) |
| 66% Tibia muscle CSA | | | | | |
| Baseline, cm² | 123 | 66.3 ± 11.3 | 121 | 66.5 ± 10.2 | |
| % Δ 4 months | 107 | 1.6 (1.2, 2.0) † | 108 | 0.7 (0.2, 1.2) ** | 0.9 (0.3, 1.6) |
| 66% Tibia muscle density | | | | | |
| Baseline, mg/cm³ | 123 | 76.9 ± 2.1 | 121 | 76.8 ± 1.9 | |
| % Δ 4 months | 107 | 0.5 (0.2, 0.7) † | 108 | 0.2 (-0.1, 0.5) | 0.3 (-0.1, 0.7) |

Baseline values are means ± SD and within and between group differences are unadjusted absolute or percentage changes with 95% Cl. Net differences (95% Cl) were calculated by subtracting within group changes from baseline for the fortified drink group from the within group changes in the placebo group. * P<0.05, ** P<0.01, † P<0.001 within-group change relative to baseline; P<0.01 between-group difference for the change relative to baseline (group-by-time interaction).

### 2.9 Physical Activity

Results are shown in Table 13.

Table 13

SEDENTARY TIME, LIGHT INTENSITY PHYSICAL ACTIVITY (PA) AND MODERATE-VIGOROUS PA

| Characteristics | n | Fortified drink | n | Placebo drink | Between-Group Difference (95% Cl) |
|---|---|---|---|---|---|
| Sedentary time | | | | | |
| Baseline, minutes | 123 | 679 ± 69 | 121 | 677 ± 73 | - |
| Δ 2 months | 109 | -11 (-23, 1.0) | 108 | -9 (-24, 4) | -2 (-20, 17) |
| Δ 4 months | 108 | -20 (-30, -9) ** | 108 | -6 (-16, 5) | -14 (-29, 1) |
| Light intensity PA | | | | | |
| Baseline, minutes | 123 | 223 ± 46 | 121 | 220 ± 47 | - |
| Δ 2 months | 109 | -2 (-10, 6) | 108 | -2 (-8, 4) | 0 (-10, 10) |
| Δ 4 months | 108 | 12 (4, 20) ** | 108 | 2 (-5, 9) | 10 (-1, 20) |
| Moderate-vigorous PA | | | | | |
| Baseline, minutes | 123 | 31 ± 16 | 121 | 31 ± 15 | - |
| Δ 2 months | 109 | 4.7 (2.1, 7.2) † | 108 | 2.9 (0.4, 5.5) * | 1.8 (-1.8, 5.3) |
| Δ 4 months | 108 | 5.2 (2.1, 8.3) † | 108 | 1.2 (-1.5, 3.9) | 4.0 (-0.1, 8.1) |

Baseline values are means ± SD and within and between group differences are unadjusted absolute changes with 95% Cl. Net differences (95% Cl) were calculated by subtracting within group changes from baseline for the fortified drink group from the within group changes in the placebo group. ** P<0.01, † P≤ 0.001 within-group change relative to baseline; P<0.05 between-group difference for the change relative to baseline (group-by-time interaction).

### 2.10 Vitality

Results are shown in Table 14.

Table 14

CHALDER FATIGUE SCALE (CFS) AND THE SUBJECTIVE VITALITY SCALE (SVS) SCORES

| Characteristics | n | Fortified drink | n | Placebo | Between-Group Differences (95% Cl) |
|---|---|---|---|---|---|
| CFQ - Total | | | | | |
| Baseline | 123 | 12.6 ± 3.5 | 120 | 12.9 ± 3.9 | - |
| Δ 4 months | 107 | -2.5 (-3.4, -1.7) † | 107 | -2.2 (-3.3, -1.1) † | -0.3 (-1.7, 1.1) |
| CFQ - Physical fatigue | | | | | |
| Baseline | 123 | 8.2 ± 2.4 | 120 | 8.3 ± 2.7 | - |
| Δ 4 months | 107 | -1.9 (-2.6, -1.3) † | 107 | -1.5 (-2.3, -0.8) † | -0.4 (-1.4, 0.6) |
| CFQ - Mental fatigue | | | | | |
| Baseline | 123 | 4.4 ± 1.5 | | 4.7 ± 1.6 | - |
| Δ 4 months | 107 | -0.6 (-0.9, -0.2) ** | 107 | -0.7 (-1.1, -0.2) ** | 0.1 (-0.5, 0.6) |
| SVS | | | | | |
| Baseline | 123 | 24.3 ± 7.6 | 121 | 24.8 ± 8.3 | - |
| Δ 4 months | 107 | 3.5 (2.1, 5.0) † | 107 | 2.5 (1.2, 3.8) † | 1.0 (-0.9, 2.9) |

Baseline values are means ± SD and within and between group differences are unadjusted absolute changes with 95% Cl. Net differences (95% Cl) were calculated by subtracting within group changes from baseline for the fortified drink group from the within group changes in the placebo group. ** P<0.01; † P<0.001 within-group change relative to baseline.

### INDUSTRIAL APPLICATION

The methods and compositions described herein are useful for maintaining or increasing mobility and/or vitality in a subject.

Where in the foregoing description reference has been made to elements or integers having known equivalents, then such equivalents are included as if they were individually set forth.

Although the invention has been described by way of example and with reference to particular embodiments, it is to be understood that modifications and/or improvements may be made without departing from the scope or spirit of the invention.

1-33. (canceled)

34. A method of maintaining or increasing mobility and/or vitality in a subject, the method comprising administration of a composition comprising milk fat globule membrane material comprising one or more polar lipids to a subject.

35. The method of claim 34, wherein maintaining or increasing mobility comprises
(a) maintaining or increasing physical performance,
(b) maintaining or reducing net bone loss and/or treating or preventing a condition associated with net bone loss,
(c) maintaining or increasing body tone, or
(d) any combination of any two or more of (a) to (c).

36. The method of claim 35, wherein maintaining or increasing physical performance comprises
(a) maintaining or increasing muscle power,
(b) maintaining or increasing muscle function,
(c) maintaining or increasing balance,
(d) maintaining or increasing flexibility,
(e) maintaining or increasing aerobic fitness,
(f) maintaining or increasing aerobic endurance,
(g) maintaining or increasing sports performance, or
(h) any combination of any two or more of (a) to (g).

37. The method of claim 35, wherein maintaining or increasing body tone comprises
(a) maintaining or increasing total body lean muscle mass,
(b) maintaining or increasing trunk lean muscle mass,
(c) maintaining or reducing total body fat mass,
(d) maintaining or reducing trunk fat mass, or
(e) any combination of any two or more of (a) to (d).

38. The method of claim 34, wherein the composition is formulated to provide at least about 200 mg of the one or more polar lipids per day.

39. The method of claim 34, wherein the composition is
(a) a nutritional composition, or
(b) a beverage, bar, gel, shot, cultured milk, UHT milk, yoghurt, custard, ice cream, cheese, crisps, chocolate, multi-layered bites, a supplement, a tablet, a capsule, confectionery or milk powder.

40. The method of claim 39, wherein the beverage is a fruit juice, a sports drink or a milk drink.

41. The method of claim 34, wherein the composition is a pharmaceutical composition comprising a pharmaceutically acceptable adjuvant or carrier.

42. The method of claim 41, wherein the composition is formulated to provide or is administered separately, simultaneously or sequentially with one or more of
(a) at least about 5 g protein per day,
(b) from about 0.1 to about 10 g lipid per day,
(c) at least about 0.75 g calcium per day, and
(d) at least about 7.5 µg vitamin D per day.

43. The method of claim 34, wherein the composition is formulated to provide or is administered separately, simultaneously or sequentially with one or more of
(a) at least about 5 g protein per day,
(b) from about 0.1 to about 10 g lipid per day,
(c) at least about 0.75 g calcium per day,
(d) at least about 7.5 µg vitamin D per day, or
(e) any combination of any two or more of (a) to (d).

44. The method of claim 34, wherein the composition comprises at least about 0.3 g of the one or more polar lipids per 100 g of the composition on a dry basis.

45. The method of claim 34, wherein the composition comprises on a dry basis
(a) at least about 5% by weight protein,
(b) from about 0.01 to about 20% by weight lipid,
(c) at least about 1% by weight calcium,
(d) at least about 10 µg vitamin D per 100 g of the composition, or
(e) any combination of any two or more of (a) to (d).

46. The method of claim 45, wherein the composition comprises on a dry basis
(a) at least about 5% by weight protein,
(b) from about 0.01 to about 20% by weight lipid,
(c) at least about 1% by weight calcium, and
(d) at least about 10 µg vitamin D per 100 g of the composition.

47. The method of claim 34, wherein the one or more polar lipids comprise one or more phospholipids, one or more gangliosides, one or more ceramides, one or more cerebrosides, one or more sphingolipids or any combination of any two or more thereof.

48. The method of claim 47, wherein
(a) the one or more gangliosides comprises GD3, GM3 or a combination thereof;
(b) the one or more phospholipids comprises one or more phosphatidylcholines, one or more phosphatidylinositols, one or more phosphatidylserines, one or more lysophospholipids, one or more phosphatidylethanolamines, one or more sphingomyelins, one or more dihydrosphingomyelins, one or more glycerophospholipids, phosphatidylglycerol, or any combination of any two or more thereof;
(c) the one or more lysophospholipids comprises lysophosphatidylcholine, lysophosphatidylserine, lysophosphatidylethanolamine or any combination of any two or more thereof;
(d) the one or more sphingomyelins comprises sphingomyelin, dihydrosphingomyelin or a combination thereof; and/or
(e) the one or more glycerophospholipids comprises phosphatidylglycerol.

49. The method of claim 34, wherein the composition comprises one or more strains of probiotic microorganisms.

50. The method of claim 34, wherein the composition is administered within about 16 hours of the subject undertaking exercise.

51. The method of claim 34, wherein
(a) the subject is aged 45 to 65 years;
(b) the subject is female;
(c) the subject is sedentary for at least about 6 hours per day; and/or
(d) the subject is undertaking exercise on at least two days per week.

52. The method of claim 34, comprising administration of the composition at least 1 or 2 times per day for a period of at least about three months.

53. The method of claim 34, wherein the composition maintains or increases mobility and/or vitality in a subject after less than about six, four or three months of commencement of administration of the composition.
